(19) United States
(12) Patent Application Publication
(10) Pub. No.: US 2020/0316188 A1
(43) Pub. Date: Oct. 8, 2020

(54) BROAD AND LONG-LASTING INFLUENZA VACCINE

(71) Applicant: Altimmune, Inc, Gaithersburg, MD (US)

(72) Inventors: Scot Roberts, Gaithersburg, MD (US); Sybil Tasker, Gaithersburg, MD (US)

(73) Assignee: Altimmune, Inc, Gaithersburg, MD (US)

(21) Appl. No.: 16/840,723

(22) Filed: Apr. 6, 2020

Related U.S. Application Data
(60) Provisional application No. 62/830,444, filed on Apr. 6, 2019.

(51) Int. Cl. A61K 39/145; A61K 9/00; A61P 31/16

(52) U.S. Cl. CPC A61K 39/145; A61P 31/16; A61K 9/0043

(57) ABSTRACT

Provided herein are monovalent pharmaceutical compositions (vaccine compositions) and methods for inducing a multi-arm (mucosal, humoral and cell-mediated) immune response and extended seroprotection of at least 12 months post vaccination against influenza virus.

### RELATED APPLICATIONS

This application claims priority to U.S. Ser. No. 62/830,442 filed on 6 Apr. 2019, which is incorporated herein in its entirety.

### FIELD OF THE DISCLOSURE

This application pertains generally to a monovalent influenza pharmaceutical formulation for intranasal administration that induces a combined mucosal, humoral and cell-mediated protective immune response in human subjects and provides seroprotection against Influenza A and Influenza B subtypes for an extended period of time.

### BACKGROUND OF THE DISCLOSURE

Influenza is one of the most common viral respiratory infections, leading to significant morbidity and mortality. The US Centers for Disease Control and Prevention recommends that everyone in the US over 6 months of age receives an annual influenza vaccination. Vaccine effectiveness can vary greatly from year to year, and in many years overall protection is poor.

Influenza viruses are enveloped ribonucleic acid viruses belonging to the family of Orthomyxoviridae and are divided into three distinct types on the basis of antigenic differences of internal structural proteins (Lamb R A, Krug R M. Orthomyxoviridae: The Viruses and Their Replication. In: Fields Virology, Editors-in-Chief: Knipe D M and Howley P M. 4th Edition. Philadelphia, Pa.: Lippincott Williams and Wilkins, Publishers; 2001; 1487-1531). Two influenza types, Type A and B, are responsible for yearly epidemic outbreaks of respiratory illness in humans and are further classified based on the structure of two major external glycoproteins, hemagglutinin (HA) and neuraminidase (NA). Type B viruses, which are largely restricted to the human host, have a single HA and NA subtype. In contrast, numerous HA and NA Type A influenza subtypes have been identified to date. Type A strains infect a wide variety of avian and mammalian species.

Type A and B influenza variant strains emerge as a result of frequent antigenic change, principally from mutations in the HA and NA glycoproteins. These variant strains may arise through one of two mechanisms: selective point mutations in the viral genome [Palese P, Garcia-Sastre A. Influenza vaccines: present and future. The Journal of Clinical Investigation. 2002; 110:9-13; Nakajima S, Nobusawa E, Nakajima K. Variation in response among individuals to antigenic sites on the HA protein of human influenza virus may be responsible for the emergence of drift strains in the human population. Virology. 2000; 274:220-231] or from reassortment between two co-circulating strains [Holmes E C, Ghedin E, Miller N, Taylor J, Bao Y, St. George K, Grenfell B T, Salzberg S L, Fraser C M, Lipman D J, Taubenberger J K. Whole-genome analysis of human influenza A virus reveals multiple persistent lineages and reassortment among recent H3N2 viruses. PLoS Biology. 2005; 3:1579-1589; Barr I G, Komadina N, Hurt A C, Iannello P, Tomasov C, Shaw R, Durrant C, Sjogren H, Hampson A W. An influenza A(H3) reassortant was epidemic in Australia and New Zealand in 2003. Journal of Medical Virology. 2005; 76:391-397].

Since 1977, influenza A virus subtypes H1N1 and H3N2, and influenza B viruses have been in global circulation in humans. The current U.S. licensed inactivated trivalent and quadrivalent (containing two strain lineages of influenza B virus) vaccines are formulated to prevent influenza illness caused by these influenza viruses. Because of the frequent emergence of new influenza variant strains, the antigenic composition of influenza vaccines need to be evaluated yearly, and the influenza vaccines are reformulated almost every year. The immune response elicited by previous vaccination may not be protective against new variants.

Changes in influenza virus formulation and manufacturing are essential to protect the public from the significant morbidity and mortality associated with seasonal influenza infections. The National Institute of Allergy and Infectious Diseases (NIAID) recently developed a strategic plan to guide basic research and development of more effective influenza vaccines. [Erbelding E J, et al. A universal influenza vaccine: The strategic plan for the National Institute of Allergy and Infectious Diseases. J Infect Dis. 2018; 218:347-54] This plan highlighted multiple weaknesses of currently available vaccines, including strain mismatch exacerbated by egg passage, inadequate durability of immune response, poor cellular immune responses, and inadequate tissue-resident immunity [Erbelding et al., 2018]. Vaccines produced in eggs are not only more likely to be antigenically dissimilar the corresponding strains in circulation than vaccine produced in tissue culture [Seqirus presents favorable outcomes data for adjuvanted trivalent influenza vaccine (FLUAD®) at 6th annual IDWeek. Seqiris Web site] but are also associated with longer manufacturing timelines and supply chain risks and induce allergic response in many individuals.

Most currently licensed vaccines are based on circulating influenza strains adapted to grow in chicken eggs. In general, these vaccines are well tolerated but provide limited protection to influenza viruses that are not well matched to the vaccine strains. An intranasal live attenuated influenza virus (LAIV) vaccine has been licensed since 2003, but its use is limited to older children and adults up to age 49 years. Intranasal vaccines may be preferred over parenteral vaccines due to the ease of administration and decreased discomfort associated with administration. A significant fraction of the US population manifests fear of needles (McLenon J. et al. The fear of needles: A systematic review and meta-analysis (2019; January) J. Adv. Nurs.; 75(1):30-42). Recent post-marketing studies have shown declining effectiveness, and it was not recommended for use in the 2016-2017 and 2017-2018 influenza seasons (CDC website). While data from 2010-2011 through 2016-2017 indicated that LAIV lacked effectiveness among 2 through 17-year-olds against H1N1pdm09 influenza viruses (2009 H1N1) in the U.S., LAIV was effective against influenza B viruses, and was similarly effective against H3N2 viruses as inactivated influenza vaccines. For the 2018-2019 season, the manufacturer of LAIV4 included a new H1N1 vaccine component. Some data suggest this will result in improved effectiveness of LAIV4 against H1N1. However, no published effectiveness estimates for this vaccine component against H1N1 viruses are yet available.

Moreover, a recently published study of the 2013-2014 LAIV vaccine showed weak systemic antibody responses [King J P, McLean H Q, Meece J K, et al. Vaccine failure and serologic response to live attenuated and inactivated influenza vaccines in children during the 2013-2014 season. Vaccine. 2018; 36:1214-9]. For all types of influenza vaccines, effectiveness can vary greatly from year to year, and in many years overall protection is poor. According to the CDC, the average overall adjusted vaccine effectiveness for influenza seasons has been approximately 40% from 2005 to 2015 and was <20% in the 2014-2015 influenza season.

Cellular immunity to influenza may reduce disease severity and contagiousness in those infected, mucosal immune responses provide protection against influenza at the initial site of infection and both may be able to protect against infection in the absence of seroprotective levels of serum HAI antibody [Gould V M W, Francis J N, Anderson K J, et al. Nasal IgA provides protection against human influenza challenge in volunteers with low serum influenza antibody titre. Front Microbiol. 2017; 8:900; McMichael A J, Gotch F M, Noble G R, et al. Cytotoxic T-cell immunity to influenza. N Engl J Med. 1983; 309:13-7; Seibert C W, Rahmat S, Krause J C, et al. Recombinant IgA is sufficient to prevent influenza virus transmission in guinea pigs. J Virol. 2013; 87:7793-804; Wilkinson T M, Li C K, Chui C S, et al. Preexisting influenza specific CD4+ T cells correlate with disease protection against influenza challenge in humans. Nat Med. 2012; 18:274-80]. However, no single influenza vaccine induces those combined responses of the immune system arms to provide long term seroprotection against influenza A and influenza B subtypes. Therefore, a need remains for an influenza vaccine that induces long term seroprotection, along with a combined immune response of the mucosal, humoral and cell-mediate types.

### SUMMARY OF THE DISCLOSURE

Herein some embodiments provided include compositions and methods for inducing long term systemic immune protection against influenza A and influenza B virus subtypes in human subjects.

In embodiments provided herein is a monovalent influenza pharmaceutical formulation suitable for a single dose intranasal administration to a human subject. In certain embodiments, the formulation comprises an effective amount of at least 10¹¹ viral particle (vp) of replication deficient adenovirus vector that contains and expresses influenza virus hemagglutinin antigen codon optimized for the human subject, wherein the effective amount induces a combined mucosal, humoral (i.e., ex-mucosal antibodies, such as in blood or serum), and T cell immune response that is preferably protective from influenza infection; and, a pharmaceutically acceptable diluent or carrier. In embodiments, the formulation is configured to provide seroprotection to the human subject of an HAI antibody titer ≥40 for at least 12 months against the influenza virus.

In certain embodiment provided herein is influenza pharmaceutical formulation suitable for a single dose intranasal administration to a human subject, wherein the formulation comprises an effective amount of at least 10⁹ viral particles (vp) of replication deficient adenovirus vector that contains and expresses influenza virus hemagglutinin antigen codon optimized for the human subject, wherein the effective amount induces a combined mucosal and humoral immune response that is preferably protective, preferably being configured to provide seroprotection to the human subject of an HAI antibody titer ≥40 for at least 12 months against the influenza virus; and, a pharmaceutically acceptable diluent or carrier. In certain embodiments, the HAI antibody titer is at least 50.

In certain embodiments, the influenza virus hemagglutinin antigen is from an Influenza A virus. In embodiments, the Influenza A virus is subtype H1N1.

In certain other embodiments provided herein are method of inducing a combined mucosal, humoral and T cell immune response that is preferably protective in a human subject against influenza virus. In preferred embodiments, the methods comprise administering intranasally to a human subject a single dose of the present influenza pharmaceutical formulation, wherein the administration induces serum antibodies, mucosal antibodies and T cells against influenza virus whereby the human subject is seroprotected for at least 12 months.

In preferred embodiments, the seroprotection lasts at least 13 months, at least 14 months, or longer. In embodiments, administration induces an HAI antibody titer of at least 50 for at least 12 months post administration.

In some embodiments, the present disclosure provides methods that provide a combined mucosal, humoral and T cell immune response that is preferably protective against Influenza A virus. In embodiments, the Influenza virus A is subtype H1N1 and/or H3N2. In embodiments, the present methods provide a combined mucosal, humoral and T cell immune response that is preferably protective against Influenza B virus. In certain embodiments, the present methods provide a combined mucosal, humoral and T cell immune response that is preferably protective against Influenza A virus subtypes and Influenza B virus infection.

### BRIEF DESCRIPTION OF THE DRAWINGS

The accompanying drawings, which are incorporated into and constitute a part of this specification, illustrate one or more embodiments of the present disclosure and, together with the detailed description and examples sections, serve to explain the principles and implementations of the disclosure.

FIG. 1 shows the schematic diagram of the adenoviral vector containing the influenza virus HA gene, wherein the numbers refer to base pair number in wild-type Ad5 sequence, GenBank ID AY339865.1.

FIG. 2 shows the serum antibody (hemagglutination-inhibiting antibody, HAI) (humoral) response at day 29 post administration induced following single administration of the present monovalent influenza vaccine composition with 100% seroprotection at two dose levels, wherein the antibodies are sufficient to prevent an infection from influenza virus. Fluzone is an inactivated quadrivalent high dose seasonal influenza vaccine.

FIG. 3 shows in table format the serum antibodies (HAI, MN) measured at day 29 post administration of the present monovalent influenza vaccine composition, wherein serum neutralizing antibodies were measured in a microneutralization (“MN”) assay and the serum hemagglutination inhibiting antibodies measured in a HAI assay are presented as a geometric mean titer (“GMT”).

FIG. 4 shows T cell immunity (cell mediated immune) response induced following single administration of the present monovalent influenza vaccine composition.

FIG. 5 shows mucosal IgA antibody (mucosal) response induced following single administration of the present monovalent influenza vaccine composition.

FIG. 6 shows extended seroprotection via measurement of serum antibody (HAI) at amounts sufficient to prevent an infection from influenza virus. The figure shows the geometric mean (95% Confidence Interval) hemagglutination inhibition titer against influenza A/California/07/2009(H1N1) to end point day 181 by dose of the present monovalent influenza vaccine composition.

FIG. 7 shows geometric mean (GMR) (95% confidence interval) microneutralization titer against Influenza A/California/07/2009(H1N1) to end point day 181 by dose of the present monovalent influenza vaccine composition.

FIG. 8 shows the cellular immune response to the present monovalent influenza vaccine composition by dose. Abbreviations: CI=confidence interval; GM=geometric mean titer; LS=least squares; SFU=spot-forming units; vp=viral particles. a. The analysis of covariance uses log-transformed level as dependent variable, dose group as a factor, and baseline log-transformed analysis as a covariate. Differences of LS mean estimates and 95% CIs were back-transformed to the original scale, resulting in a ratio of the geometric means; b. Post-hoc analysis; c. The number and percentage of subjects with 3-fold rise since Baseline and 25 SFU/10⁶ cells greater than Baseline; and, d. From Fisher's exact test.

FIG. 9 shows humoral immune response induced by the present monovalent influenza vaccine composition (e.g., NasoVAX) at a dose of 11×10¹¹ vp and Fluzone® Groups on days 29, 91 and 181 post administration, with the HAI presented as a geometric mean ratio (“GMR”) along with the seroconversion rate (“SCR”) and the percentage of subjects with a HAI titer ≥1:40 (“a”), and the seroprotection rate (“SPR”), wherein the percentage of subjects with either a baseline HAI titer, 1:10 and postvaccination titer≥1:40 (“b) (which is 4 times the assay lower limit of quantification) are included in the last row.

FIG. 10A shows the HAI titer (“GMT”) over 13 months induced by the present monovalent influenza vaccine composition (e.g., NasoVAX), wherein 8/15 subjects in the high dose group (11×10¹¹ vp) returned at about 13 months for evaluation with an average of 13.5 months from administration to measurement of HAI titer.

FIG. 10B shows seroprotection and seroconversion rates induced by the present monovalent influenza vaccine composition (e.g., NasoVAX), demonstrating the seroprotection and seroconversion rates are identical between study days 15 and 400 (days post administration of the present monovalent influenza vaccine composition). The immune response was intact at 13 months with the rate of seroprotection and the rate of seroconversion unchanged.

FIG. 11 shows a dose-dependent vector shedding that is absent at 2 weeks post-administration (of the present monovalent influenza vaccine composition (e.g., NasoVAX)) with no replication competent virus found (as determined via polymerase chain reaction (“PCR”) assay) and anti-vector antibody presented as GMR at Day 29 vs baseline wherein only a 2.3-fold induction after 1 month at highest dose was demonstrated. The present monovalent influenza vaccine composition demonstrates a transient shedding (Advector) with limited anti-vector (Ad-vector) immune response.

FIG. 12 shows the effect on NasoVAX immunogenicity (high dose; 11×10¹¹ vp) of pre-existing anti-vector (Ad5) immunity as measured for humoral (“HAI” or microneutralization “MN” at day 29), mucosal (“IgA” at day 29) and cellular (“ELISpot” at day 8) wherein no difference in an immune response between Ad5 seronegative or Ad5 seropositive subjects was observed. Median titer of Ad5+ subjects (seropositive) was 22-fold above the lower limit of quantification (LLOQ), wherein seroconversion is typically about 4-fold over background or the LLOQ assay.

### DETAILED DESCRIPTION OF THE INVENTION

### Introduction

The present invention provides compositions and methods for inducing long term systemic immune protection against influenza A virus subtypes and influenza B viruses in human subjects. In certain embodiments provided herein is a influenza pharmaceutical formulation (preferably monovalent) suitable for a single dose intranasal administration to a human subject. In embodiments, the formulation comprises an effective amount of at least 10¹¹ viral particle (vp) of replication deficient adenovirus vector that contains and expresses influenza virus hemagglutinin antigen codon optimized for the human subject, wherein the effective amount induces a combined mucosal, humoral and T cell immune response, which is preferably protective against influenza infection; and, a pharmaceutically acceptable diluent or carrier.

In other embodiments, is provided an influenza pharmaceutical formulation suitable for a single dose intranasal administration to a human subject, wherein the formulation comprises an effective amount of at least 10⁹ viral particles (vp) of replication deficient adenovirus vector that contains and expresses influenza virus hemagglutinin antigen codon optimized for the human subject, wherein the effective amount induces a combined mucosal and humoral immune response, which is preferably protective against influenza infection, and is some preferred embodiments configured to provide seroprotection to the human subject of an HAI antibody titer ≥40 for at least 12 months against the influenza virus; and, a pharmaceutically acceptable diluent or carrier

In certain embodiments is provided a method for inducing a combined mucosal, humoral and T cell immune response, preferably protective, in a human subject against influenza virus. In certain embodiments, the methods comprise administering intranasally to a human subject a single dose of a present influenza pharmaceutical formulation, wherein the administration induces serum antibodies, mucosal antibodies and T cells against influenza virus whereby the human subject is seroprotected for at least 12 months.

Applicants have developed an adenoviral vector (e.g., Ad5-vectored), intranasal influenza vaccine produced in tissue culture (NasoVAX). Adenovirus is a naturally occurring respiratory virus that has been used frequently as a vector to introduce genetic material into cells. By incorporating the influenza HA gene into replication-deficient (RD) adenovirus (Ad-HA) and applying the Ad-HA into the nose (intranasal route of administration), the adenoviral vector can transduce the HA gene into cells of the nasal mucosa, leading to transient expression of the encoded HA protein. NasoVAX is delivered intranasally, where we herein demonstrate the vaccine composition induced both local and long-lasting systemic immune responses. See Example 3. Subsequent production of the HA antigen in normal human epithelial cells allows for an immune response against the HA antigen as it occurs in natural circulating influenza virus. Moreover, Applicants have shown the use of the adenoviral vector, when administered intranasally, even in subjects seropositive for Ad5, bypasses the adenovirus immunity of the subjects and thereby its effects are not adversely affected by a pre-existing immune response against the vector. See FIG. 13. In embodiments, the present intranasal influenza vaccine (comprising an adenoviral vector) can be administered repeatedly (e.g., as a seasonal influenza vaccine administered about once every 11-14 months) without inducing a significant immune response against the viral vector.

The clinical study disclosed herein was designed to evaluate the safety and immunogenicity of a monovalent A/California/04/2009(H1N1)-like strain version of NasoVAX. Exploratory endpoints were included to evaluate the breadth of antibody response and the ability of NasoVAX to induce cellular and mucosal immune responses. See Example 2 and 3. The trial results disclosed herein demonstrate the present monovalent influenza pharmaceutical formulation satisfies the unmet needs identified by NIAID [Erbelding et al., 2018].

As understood by one of skill in the art, the measurement of HAI antibodies is used as a surrogate of protection wherein a HAI antibody titer of ≥40 measured in post vaccination serum demonstrates induced seroprotection by the administered influenza vaccine [Trombetta C M. et al.; Overview of Serological Techniques for Influenza Vaccine Evaluation: Past, Present and Future Vaccines (Basel) (2014) Dec. 2(4): 707-734]. A “surrogate of protection” means an immune marker that can substitute for the clinical end point and thus, can be used to reliably predict vaccine efficacy. In the case of influenza vaccines, antibodies measured in a hemagglutination inhibition (HAI) assay is a surrogate of protection, wherein the HAI assay is based on the ability of antibodies, if present in the serum, to prevent agglutination between erythrocytes and viral hemagglutinin. It is generally understood that subjects with a HAI antibody titer of ≥40 are protected from represented Influenza A virus subtypes and Influenza B virus. In other words, an HAI antibody titer of 40 (or greater) is generally considered as a “protective” threshold level, beyond which there is a 50% or greater reduction in the possibility of contracting an influenza infection. An HAI titer equal to or greater than 40 is used as an immunological correlate of protection and is regarded as the best currently available parameter for predicting protection from natural infection, according to FDA guidelines for pandemic influenza vaccines (Noah et al. Qualification of the hemagglutination inhibition assay in support of pandemic influenza vaccine licensure. Clin. Vaccine Immunol. 2009; 16:558-566).

In a separate study, Applicants have shown the present intranasal influenza vaccine (NasoVAX) is stable for about 3 months at an ambient temperature, such as room temperature (e.g., 15 to 30° C., preferably 20-25° C.). See Example 7. In embodiments, the present intranasal influenza vaccine can be stored, or shipped, without the need for refrigeration or specific storage conditions. In certain embodiments, the present intranasal influenza vaccine comprises influenza antigens present in an Influenza A pandemic virus strain and may be shipped directly to the user i.e., vaccine, for intranasal administration.

### Definitions

As used herein, the terms “a” or “an” are used, as is common in patent documents, to include one or more than one, independent of any other instances or usages of “at least one” or “one or more.”

As used herein, the term “or” is used to refer to a nonexclusive or, such that “A or B” includes “A but not B,” “B but not A,” and “A and B,” unless otherwise indicated.

As used herein, the term “about” is used to refer to an amount that is approximately, nearly, almost, or in the vicinity of being equal to or is equal to a stated amount, e.g., the state amount plus/minus about 5%, about 4%, about 3%, about 2% or about 1%.

The compositions, formulations and methods of the present invention may comprise, consist essentially of, or consist of the components and ingredients of the present invention as well as other ingredients described herein. As used herein, “consisting essentially of” means that the compositions, formulations and methods may include additional steps, components or ingredients, but only if the additional steps, components or ingredients do not materially alter the basic and novel characteristics of the claimed compositions, formulations and methods.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

As used herein, an “adjuvant” refers to a substance that enhances the body's immune response to an antigen. In embodiments, the present monovalent influenza pharmaceutical formulation is a non-adjuvanted vaccine composition.

By “administration” is meant introducing a vaccine composition of the present disclosure into a subject; it may also refer to the act of providing a composition of the present disclosure to a subject (e.g., by prescribing). The term “therapeutically effective amount” as used herein refers to that amount of the compound being administered which will induce a combined, mucosal, humoral and cell mediated immune response. The term also refers to an amount of the present compositions that will relieve or prevent to some extent one or more of the symptoms of the condition to be treated. In reference to conditions/diseases that can be directly treated with a composition of the disclosure, a therapeutically effective amount refers to that amount which has the effect of preventing the condition/disease from occurring in a mammal that may be predisposed to the disease but does not yet experience or exhibit symptoms of the condition/disease (prophylactic treatment), alleviation of symptoms of the condition/disease, diminishment of extent of the condition/disease, stabilization (e.g., not worsening) of the condition/disease, preventing the spread of condition/disease, delaying or slowing of the condition/disease progression, amelioration or palliation of the condition/disease state, and combinations thereof. The term “effective amount” refers to that amount of the compound being administered which will produce a reaction that is distinct from a reaction that would occur in the absence of the compound.

In embodiments, an effective amount of the present monovalent influenza pharmaceutical formulation comprises at least 10⁹ infectious units (ifu) of a replication deficient adenoviral vector containing and expressing influenza virus hemagglutinin antigen codon optimized for the human subject.

As used herein, the term “ambient temperature” is the air temperature for storing the present monovalent influenza pharmaceutical formulation. In embodiments, the ambient temperature is a room temperature, such as selected from any temperature within the range from about 15 to 30° C., preferably from about 20 to 25° C.

As used herein, the term “human adenovirus” is intended to encompass all human adenoviruses of the Adenoviridae family, which include members of the Mastadenovirus genera. To date, over fifty-one human serotypes of adenoviruses have been identified (see, e.g., Fields et al., Virology 2, Ch. 67 (3d ed., Lippincott-Raven Publishers)). The adenovirus may be of serogroup A, B, C, D, E, or F. The human adenovirus may be a serotype 1 (Ad 1), serotype 2 (Ad2), serotype 3 (Ad3), serotype 4 (Ad4), serotype 5 (Ad5), serotype 6 (Ad6), serotype 7 (Ad7), serotype 8 (Ad8), serotype 9 (Ad9), serotype 10 (Ad10), serotype 11 (Ad11), serotype 12 (Ad2), serotype 13 (Ad13), serotype 14 (Ad14), serotype 15 (Ad15), serotype 16 (Ad16), serotype 17 (Ad17), serotype 18 (Ad8), serotype 19 (Ad9), serotype 19a (Ad19a), serotype 19p (Ad19p), serotype 20 (Ad20), serotype 21 (Ad21), serotype 22 (Ad22), serotype 23 (Ad23), serotype 24 (Ad24), serotype 25 (Ad25), serotype 26 (Ad26), serotype 27 (Ad27), serotype 28 (Ad28), serotype 29 (Ad29), serotype 30 (Ad30), serotype 31 (Ad31), serotype 32 (Ad32), serotype 33 (Ad33), serotype 34 (Ad34), serotype 35 (Ad35), serotype 36 (Ad36), serotype 37 (Ad37), serotype 38 (Ad38), serotype 39 (Ad39), serotype 40 (Ad40), serotype 41 (Ad41), serotype 42 (Ad42), serotype 43 (Ad43), serotype 44 (Ad44), serotype 45 (Ad45), serotype 46 (Ad46), serotype 47 (Ad47), serotype 48 (Ad48), serotype 49 (Ad49), serotype 50 (Ad50), serotype 51 (Ad51), or combinations thereof, but are not limited to these examples. In certain embodiments, the adenovirus is serotype 5 (Ad5).

As used herein, a “pharmaceutically acceptable carrier” refers to a carrier or diluent that does not cause significant irritation to the human subject and does not abrogate the biological activity and properties of the administered vaccine compositions.

As used here, the term “seroconversion” is a rate defined as the percentage of individuals vaccinated (administered a present vaccine formulation) who have at least a 4-fold increase in serum haemagglutinin inhibition (HI) titers after vaccination. As used herein “conversion factor” is defined as the fold increase in serum HI geometric mean titers (GMTs) after vaccination.

As used herein, the term “seroprotection” refers to an HAI antibody titer of 40 or greater measured in serum from a human subject post-vaccination. The term “protection rate” as used herein is defined as the percentage of individuals vaccinated with a serum HAI titer equal to or greater than 1:40 after vaccination and is normally accepted as indicating protection.

As used herein, the term “seasonal influenza virus” refers to an influenza A and/or B virus that circulates and are responsible for seasonal flu epidemics each year. Influenza A viruses are divided into subtypes based on two proteins on the surface of the virus: hemagglutinin (HA) and neuraminidase (NA). There are 18 different hemagglutinin subtypes and 11 different neuraminidase subtypes (H1 through H18 and N1 through N11, respectively). While there are potentially 198 different influenza A subtype combinations, only 131 subtypes have been detected in nature. Current subtypes of influenza A viruses that routinely circulate in humans include A(H1N1) and A(H3N2). Influenza B viruses are further classified into two lineages: B/Yamagata and B/Victoria. Both influenza A and B viruses can be further classified into specific clades and sub-clades (which are sometimes called groups and sub-groups). Currently circulating influenza A(H1N1) viruses are related to the pandemic 2009 H1N1 virus that emerged in the spring of 2009 and caused a flu pandemic. This virus, referred to as the “A(H1N1)pdm09 virus,” and more generally called “2009 H1N1,” continues to circulate and contribute to seasonal flu epidemics each year. Of all the influenza viruses that routinely circulate and cause illness in people, influenza A(H3N2) viruses tend to change more rapidly, both genetically and antigenically. Influenza A(H3N2) viruses have formed many separate, genetically different clades in recent years that continue to co-circulate.

As used herein, the term “pandemic influenza virus” refers to an influenza A virus that circulates globally and is responsible for a global flu pandemic. Pandemics happen when new (novel) influenza A viruses emerge which are able to infect people easily and spread from person to person in an efficient and sustained way, spreading globally.

The terms “treat”, “treating”, and “treatment” are an approach for obtaining beneficial or desired clinical results. Specifically, beneficial or desired clinical results include, but are not limited to, alleviation of symptoms, diminishment of extent of disease, stabilization (e.g., not worsening) of disease, delaying or slowing of disease progression, substantially preventing spread of disease, amelioration or palliation of the disease state, and remission (partial or total) whether detectable or undetectable. In addition, “treat”, “treating”, and “treatment” can also mean prolonging survival as compared to expected survival if not receiving treatment and/or can be therapeutic in terms of a partial or complete cure for a disease and/or adverse effect attributable to the disease. As used herein, the terms “prophylactically treat” or “prophylactically treating” refers completely, substantially, or partially preventing a disease/condition or one or more symptoms thereof in a host. Similarly, “delaying the onset of a condition” can also be included in “prophylactically treating” and refers to the act of increasing the time before the actual onset of a condition in a patient that is predisposed to the condition.

As used herein, a “vaccine” refers to a composition comprise the adenoviral vector containing and expressing an influenza antigen, along with other components of a vaccine formulation, including for example adjuvants, slow release compounds, solvents, etc. In embodiments of the invention vaccines improve immune responses to any antigen regardless of the antigen source or its function.

As referred to herein, a “vector” carries a genetic code, or a portion thereof, for an antigen, however it is not the antigen itself. In an exemplary aspect, a vector can include a viral vector or bacterial vector. As referred to herein an “antigen” means a substance that induces a specific immune response in a subject, including humans and/or animals. The antigen may comprise a whole organism, killed, attenuated or live; a subunit or portion of an organism; a recombinant vector containing an insert with immunogenic properties; a piece or fragment of DNA capable of inducing an immune response upon presentation to a host animal; a polypeptide, an epitope, a hapten, or any combination thereof. In various aspects, the antigen is a virus, bacterium, a subunit of an organism, an auto-antigen, or a cancer antigen.

Vaccine Formulation

Provided herein are influenza pharmaceutical formulations, also referred to herein as vaccine formulations, suitable and/or configured for a single dose intranasal administration to a human subject. In embodiments, the instant formulations comprise an effective amount of at least 10⁹ viral particle (vp) of replication deficient adenovirus vector that contains and expresses influenza virus hemagglutinin antigen codon optimized for the human subject and a pharmaceutically acceptable diluent or carrier. In exemplary embodiments the formulation is a monovalent influenza pharmaceutical formulation. In certain embodiments, the adenoviral vector is present in a formulation buffer comprising 10 mM TRIS, 75 mM NaCl, 0.2% Polysorbate 80, 5% sucrose, 1 mM MgCl₂, 0.1 mM EDTA, 0.5% ethanol, 10 mM L-Histidine.

In alternative embodiments, the present replication deficient adenovirus vector that contains and expresses influenza virus hemagglutinin antigen codon optimized for the human subject, may be combined with other influenza antigens (e.g. viral vector expressed antigens) to form a multivalent influenza pharmaceutical formulation. The other components may be included to induce a humoral response with antibodies to a different epitope than that presented in the instant adenoviral vectored containing influenza virus hemagglutinin antigen. In other embodiments, the other component(s) may be included to induce a different arm of the immune system, such as cell-mediated or mucosal immune response to an influenza antigen.

In exemplary embodiments provided herein is a monovalent influenza pharmaceutical formulation suitable for a single dose intranasal administration to a human subject, comprising: an effective amount of at least 10¹¹ viral particles (vp) of replication deficient adenovirus vector that contains and expresses influenza virus hemagglutinin antigen codon optimized for the human subject, wherein the effective amount induces a combined mucosal, humoral and T cell protective immune response configured to provide seroprotection to the human subject for at least 12 months against the influenza virus; and, a pharmaceutically acceptable diluent or carrier.

In other exemplary embodiments provided herein is an influenza pharmaceutical formulation suitable for a single dose intranasal administration to a human subject, comprising an effective amount of at least 10⁹ viral particles (vp) of replication deficient adenovirus vector that contains and expresses influenza virus hemagglutinin antigen codon optimized for the human subject, wherein the effective amount induces a combined mucosal and humoral protective immune response configured to provide seroprotection to the human subject of an HAI antibody titer ≥40 for at least 12 months against the influenza virus; and, a pharmaceutically acceptable diluent or carrier.

In certain embodiments, the non-replicating adenoviral viral vector is a human adenovirus. In alternative embodiments, the adenovirus is a bovine adenovirus, a canine adenovirus, a non-human primate adenovirus, a chicken adenovirus, or a porcine or swine adenovirus. In exemplary embodiments, the non-replicating viral vector is a human adenovirus.

In embodiments, non-replicating adenoviral vectors are particularly useful for gene transfer into eukaryotic cells and vaccine development, and in animal models.

In embodiments, any adenoviral vector (Ad-vector) known to one of skill in art, and prepared for administration to a mammal, which may comprise and express an influenza antigen may be used in the compositions and with the methods of this application. Such Ad-vectors include any of those in U.S. Pat. Nos. 6,706,693; 6,716,823; 6,348,450; or US Patent Publ. Nos. 2003/0045492; 2004/0009936; 2005/0271689; 2007/0178115; 2012/0276138 (herein incorporated by reference in entirety).

In certain embodiments the recombinant adenovirus vector may be non-replicating or replication-deficient requiring complementing E1 activity for replication. In embodiments the recombinant adenovirus vector may include E1-defective, E3-defective, and/or E4-defective adenovirus vectors, or the “gutless” adenovirus vector in which viral genes are deleted. The E1 mutation raises the safety margin of the vector because E1-defective adenovirus mutants are replication incompetent in non-permissive cells. The E3 mutation enhances the immunogenicity of the antigen by disrupting the mechanism whereby adenovirus down-regulates MHC class I molecules. The E4 mutation reduces the immunogenicity of the adenovirus vector by suppressing the late gene expression, thus may allow repeated re-vaccination utilizing the same vector. In exemplary embodiments, the recombinant adenovirus vector is an E1 and E3 defective vector.

The “gutless” adenovirus vector replication requires a helper virus and a special human 293 cell line expressing both E1a and Cre, a condition that does not exist in natural environment; the vector is deprived of viral genes, thus the vector as a vaccine carrier is non-immunogenic and may be inoculated for multiple times for re-vaccination. The “gutless” adenovirus vector also contains 36 kb space for accommodating transgenes, thus allowing co-delivery of a large number of antigen genes into cells. Specific sequence motifs such as the RGD motif may be inserted into the H-I loop of an adenovirus vector to enhance its infectivity. An adenovirus recombinant may be constructed by cloning specific transgenes or fragments of transgenes into any of the adenovirus vectors such as those described below. The adenovirus recombinant vector is used to transduce epidermal cells of a vertebrate in a non-invasive mode for use as an immunizing agent. The adenovirus vector may also be used for invasive administration methods, such as intravenous, intramuscular, or subcutaneous injection.

With respect to dosages, routes of administration, formulations, adjuvants, and uses for recombinant viruses and expression products therefrom, compositions of the invention may be used for parenteral or mucosal administration, preferably by intradermal, subcutaneous, intranasal or intramuscular routes. When mucosal administration is used, it is possible to use oral, ocular or nasal routes. In exemplary embodiments, the present vaccine formulations are administered intranasally.

The formulations which comprise the adenovirus vector of interest, can be prepared in accordance with standard techniques well known to those skilled in the pharmaceutical or veterinary art. See Example 1. Such formulations can be administered in dosages and by techniques well known to those skilled in the clinical arts taking into consideration such factors as the age, sex, weight, and the route of administration. The formulations can be administered alone or can be co-administered or sequentially administered with compositions, e.g., with “other” immunological composition, or attenuated, inactivated, recombinant vaccine or therapeutic compositions thereby providing multivalent or “cocktail” or combination compositions of the invention and methods employing them. In embodiments, the formulations may comprise sucrose as a cryoprotectant and polysorbate-80 as a non-ionic surfactant. In certain embodiments, the formulations further comprise free-radical oxidation inhibitors ethanol and histidine, the metal-ion chelator ethylenediaminetetraacetic acid (EDTA), or other agents with comparable activity (e.g., block or prevent metal-ion catalyzed free-radical oxidation).

The formulations may be present in a liquid preparation for mucosal administration, e.g., oral, nasal, ocular, etc., formulations such as suspensions and, preparations for parenteral, subcutaneous, intradermal, intramuscular, intravenous (e.g., injectable administration) such as sterile suspensions or emulsions. In such formulations the adenoviral vector may be in admixture with a suitable carrier, diluent, or excipient such as sterile water, physiological saline, or the like. The formulations can also be lyophilized or frozen. The formulations can contain auxiliary substances such as wetting or emulsifying agents, pH buffering agents, adjuvants, preservatives, and the like, depending upon the route of administration and the preparation desired. The formulations can contain at least one adjuvant compound. In exemplary embodiments, the present vaccine formulations are non-adjuvanted.

Standard texts, such as “REMINGTON'S PHARMACEUTICAL SCIENCE”, 17th edition, 1985, incorporated herein by reference, may be consulted to prepare suitable preparations, without undue experimentation.

In embodiments, an effective amount (e.g., an amount that induces a combined mucosal, humoral and cell-mediated immune response) of the adenoviral vector is at least 10⁹ infectious units (ifu) of a replication deficient adenoviral vector containing and expressing influenza virus hemagglutinin antigen codon optimized for the human subject. As understood by one of skill in the art, codon optimization improves expression of heterologous genes in a host organism. The present influenza antigen was codon optimized for a mammalian host, which includes a human subject.

In certain embodiments, the present monovalent influenza formulation comprises an effective amount of about 10⁹ viral particles (vp) of a replication deficient adenoviral vector. In exemplary embodiments, the present monovalent influenza formulation comprises an effective amount of about 10¹⁰ viral particles (vp) of a replication deficient adenoviral vector. In certain other exemplary embodiments, the present monovalent influenza formulation comprises an effective amount of about 10¹¹ viral particles (vp) of a replication deficient adenoviral vector.

In embodiments, the effective amount of adenoviral vector in the present vaccine formulation induces a combined influenza-specific mucosal (as demonstrated via IgA measurement), humoral (as demonstrated via sera HAI and microneutralization antibodies) and cell mediated (as demonstrated via influenza HA antigen specific T cell activation) immune response in a human subject against influenza virus. In embodiments, the serum antibodies are seroprotective for at least 6 months, at least 12 months, at least 13 month or at least 14 months.

In embodiments, the combined immune response provides protection against Influenza A virus. In certain embodiments, the combined immune response induced by the present monovalent influenza pharmaceutical formulation provides protection against Influenza B virus. In embodiments, the Influenza A virus and/or Influenza B virus are “seasonal” influenza virus which cause seasonal epidemics, mostly during the winter months. In other embodiments, the Influenza A virus is a “pandemic” influenza virus, which can cause widespread illness due to new and different antigenic epitopes present in the virus.

Influenza A viruses are divided into subtypes based on two proteins on the surface of the virus: the hemagglutinin (H) and the neuraminidase (N). There are at least 18 different hemagglutinin subtypes and at least 11 different neuraminidase subtypes. (H1 through H18 and N1 through N11 respectively). Influenza A viruses can be further broken down into different strains. Current subtypes of influenza A viruses found in people are influenza A (H1N1) and influenza A (H3N2) viruses. Influenza B viruses are not divided into subtypes but can be further broken down into lineages and strains. Currently circulating influenza B viruses belong to one of two lineages: B/Yamagata and B/Victoria. Naming convention for influenza viruses includes multiple components and follows the approach: the antigenic type (e.g., A, B, C); the host of origin (e.g., swine, equine, chicken, etc. For human-origin viruses, no host of origin designation is given); geographical origin (e.g., Denver, Taiwan, etc.); strain number (e.g., 15, 7, etc.); year of isolation (e.g., 57, 2009, etc.); and, for influenza A viruses, the hemagglutinin and neuraminidase antigen description in parentheses (e.g., (H1N1), (H5N1).

In exemplary embodiments, the present adenoviral vector comprises a genetic insert encoding the HA surface protein antigen from an A/California/04/2009(H1N1) virus. In certain embodiments, the present adenoviral vector contains and expresses a hemagglutinin antigen from an H1N1 influenza A virus subtype. In other certain embodiments, the present adenoviral vector contains and expresses a hemagglutinin antigen from an H3N2 influenza A virus subtype. In other embodiments, the present adenoviral vector contains and expresses a hemagglutinin antigen from an influenza B virus. In certain embodiments, the present adenoviral vector contains and expresses a hemagglutinin antigen from a pandemic Influenza A virus. In certain other embodiments, the present adenoviral vector contains and expresses a hemagglutinin antigen (HA) from a seasonal influenza virus.

Methods of Use

Provided herein is a method of inducing a combined mucosal, humoral and T cell protective immune response in a human subject against influenza virus whereby the human subject is seroprotected for at least 6 months, at least 12 months, at least 13 months or longer. In embodiments, the methods comprise administering intranasally a single dose of an effective amount of at least 10⁹ viral particles (vp) of a replication deficient adenoviral vector containing and expressing influenza virus hemagglutinin antigen codon optimized for the human subject, wherein the administration induces sera antibodies, mucosal antibodies and T cells against influenza virus.

In embodiments, the present monovalent influenza pharmaceutical formulation is used to provide protection against seasonal influenza virus. In certain other embodiments, the present monovalent influenza pharmaceutical formulation is used to provide protection against pandemic influenza virus.

In embodiments, the present methods provide protection against infection by Influenza A virus subtypes. In certain embodiments, the present methods provide protection against infection by Influenza A virus subtypes H1N1 and/or H3N2. In other embodiments, the present methods provide protection against infection by Influenza B virus.

In embodiments, the seroprotection lasts at least about 13 months. In certain embodiments, the seroprotection lasts at least about 14 months, or longer.

In embodiments, the step of administering a single dose of a present (monovalent) influenza pharmaceutical formulation induces HAI antibodies at a titer of 50 or greater for at least 6 months, at least 12 months, at least 13 months, at least 14 months or longer. In embodiments the titer of HAI antibodies in a human subject 12 months post vaccination (administration of the present influenza pharmaceutical formulation) is at least 50, at least 60, at least 70, at least 80, at least 90, or at least 100.

Thus, as discussed herein and in the Examples below, NasoVAX has been surprisingly found to be seroprotective and to induce the production of neutralizing antibody response rates similar to commercial influenza vaccine (e.g. Fluzone®), an antibody response that is durable for at least one year, strong mucosal (IgA) and cellular (e.g., T cells as measured by ELISspot) responses, minimal induction of anti-vector (e.g., Ad5) antibodies, to have be little or unaffected by pre-existing vector (e.g., Ad5) antibody, to be well-tolerated at all dose levels tested, and to be stable (i.e., comprise an effective amount of viral particles) after about three months at ambient temperature (e.g., about 20-25° C.).

This disclosure, then, in some embodiments, provides influenza pharmaceutical formulations (e.g., monovalent) suitable for a single dose intranasal administration to a human subject, the formulations comprising: an effective amount such as at least about any of 10⁶ vp, 10⁷ vp, 10⁸ vp, 10⁹ vp, 10¹⁰ vp, or 10¹¹ vp, preferably at least 10⁹ vp or 10¹¹ vp of replication deficient adenovirus vector that contains and expresses influenza virus hemagglutinin antigen (HA) codon optimized for the human subject, wherein the effective amount induces a combined mucosal, humoral and T cell immune response, which is preferably protective; and, a pharmaceutically acceptable diluent or carrier. In some embodiments, the mucosal immune response, which is preferably protective either alone or in combination with the other immune responses, is determined by anti-hemagglutinin (HA) IgA ELISA, the humoral immune response, which is preferably protective either alone or in combination with the other immune responses, is determined by hemagglutination inhibition assay (HAI) titer and/or the presence of neutralizing antibody as determined using a microneutralization assay, optionally as measured using one or more of the geometric mean titer (GMT), geometric mean ratio (GMR), seroconversion rate (SCR), seropositivity rate (SPR); and/or, the T cell immune response, which is preferably protective either alone or in combination with the other immune responses, is determined by using γ-interferon ELISpot. In some embodiments, the formulation is configured to provide seroprotection to the human subject as determined by the subject having an HAI antibody titer ≥40 for at least 12 months against the influenza virus. In some embodiments, this disclosure provides pharmaceutical formulations suitable for a single dose intranasal administration to a human subject, comprising: an effective amount of at least 10⁹ viral particles (vp) of replication deficient adenovirus vector that contains and expresses influenza virus hemagglutinin antigen (HA) codon optimized for the human subject, wherein the effective amount induces a combined mucosal and humoral protective immune response configured to provide seroprotection to the human subject as determined by the subject having an HAI antibody titer ≥40 (or, in some embodiments, ≥50) for at least 12 months against the influenza virus; and, a pharmaceutically acceptable diluent or carrier. In some embodiments, the effective amount is at least about 10¹⁰ viral particles (vp) or at least about 10¹¹ viral particles (vp) and, in further embodiments, induces a T cell response. In preferred embodiments, the formulation does not comprise an adjuvant. In some embodiments, the formulation does not comprise an adjuvant. In preferred embodiments, the HA antigen is from an Influenza A virus (in some preferred embodiments subtype H1N1 or H3N2) or an Influenza B virus. In some embodiments, the formulation comprises Tris HCl (pH 7.4), histidine, sucrose, sodium chloride, magnesium chloride, polysorbate 80, ethylenediaminetetraacetic acid, and ethanol. In some embodiments, the formulation comprises a single dose, preferably an intranasal dose, of about 1×10⁹ vp, about 1×10¹⁰ vp, or about 1×10¹¹ vp. In some embodiments, the formulation is frozen. In preferred embodiments, the formulation is stable at ambient temperature for at least about three months. In embodiments, the ambient temperature is a room temperature from about 15 to 30° C., preferably from about 20 to 25° C. In some embodiments, the formulation is stored at about −20° C. or about 4-8° C. until distribution, which could require storage at ambient temperature. In some embodiments, the formulation is configured as a seasonal influenza vaccine comprising antigens from a seasonal influenza virus. In some embodiments, the formulation is configured as a pandemic influenza vaccine comprising antigens from an Influenza A pandemic virus strain. In preferred embodiments, the replication deficient adenovirus vector is human adenovirus serotype 5 (Ad5). In some embodiments, the formulation is contained within a container that, in some embodiments, can be selected from the group consisting of a glass vial, nasal sprayer, droplet dispenser, aerosolizer, and atomizer. In some embodiments, the vial can be a multi-dose vial (i.e., a vial comprising multiple doses (e.g., each dose comprising an effective dose of viral particles) of the formulation) that could be used in, e.g., pandemic situations in combination with a pipette and/or eye dropper and administered to subjects dropwise). In some embodiments, the container has contained the formulation for at least about three months at ambient temperature (e.g. room temperature). In some embodiments, the formulation is configured to contain at least about 33% (i.e., allowing for about a 0.5 log decrease (about a three-fold decrease)), preferably in some embodiments about 50%, infectious viral particles after about three months at ambient temperature (e.g. room temperature) within the container. In some embodiments, the formulation comprises at least about 33%, preferably about 50%, of the infectious viral particles present as compared to a matched formulation that has been in the same type of container at ambient temperature for less than one month (e.g., in some embodiments allowing for about a 0.5 log decrease (about a three-fold decrease)). In some embodiments, the container is a single-use container and/or configured for intranasal administration of the formulation. In some embodiments, this disclosure provides for the use of such formulations in the preparation of a medicament for administration to a human subject to prevent and/or treat infection by influenza virus in the subject. In some embodiments, this disclosure provides use of such containers in the preparation of a medicament for administration to a human subject to prevent and/or treat infection by influenza in the subject. In some embodiments, this disclosure provides kits for the preparation of preparation of a medicament for administration to a human subject to prevent and/or treat infection by influenza in the subject, said kit comprising at least one of such formulations and/or containers.

In some embodiments, this disclosure also provides methods of inducing a combined mucosal, humoral and T cell protective immune response in a human subject against influenza virus comprising: administering intranasally to a human subject at least a single dose of the influenza pharmaceutical formulation disclosed herein, wherein the administration induces a combined mucosal, humoral and T cell protective immune response against influenza virus and the human subject is seroprotected from infection by influenza virus for at least 12 months after said administration. In some embodiments, the mucosal protective immune response is determined by anti-hemagluttinin (HA) IgA ELISA, the humoral protective immune response is determined by hemagglutination inhibition assay (HAI) titer and/or presence of neutralizing antibody as determined using a microneutralization assay, optionally as measured using one or more of the geometric mean titer (GMT), geometric mean ratio (GMR), seroconversion rate (SCR), seropositivity rate (SPR); and/or, the T cell protective immune response is determined by using γ-interferon ELISpot. In some embodiments, the method(s) can include administration of multiple doses of the formulation (e.g., during an epidemic or pandemic situation). In preferred embodiments, the seroprotection lasts for at least about 13 months, or at least about 14 months. In some embodiments, the influenza virus is Influenza A (in some preferred embodiments subtype H1N1 or H3N2) and/or Influenza B virus. In some embodiments, the combined immune response provides protection against Influenza A virus subtypes and Influenza B virus. In some embodiments, the influenza virus is a seasonal influenza virus. In some embodiments, the administration induces an HAI antibody titer of at least 50 for at least 12 months post administration. In some embodiments, the subject exhibits anti-adenovirus vector immunity (preferably wherein the replication deficient adenovirus vector is human adenovirus serotype 5 (Ad5)) prior to the administering intranasally, said immunity being determined by hemagglutinin inhibition assay, microneutralization assay, IgA ELISA, and/or ELIspot assay. In preferred embodiments, administration of the formulation does not significantly enhance the anti-adenovirus vector immunity of the subject (e.g., not more than about three-fold, four-fold, five-fold or six-fold above anti-adenovirus vector immunity of the subject present before administration of the formulation), said immunity in preferred embodiments being determined by hemagglutinin inhibition assay, microneutralization assay, IgA ELISA, and/or ELIspot assay. In some embodiments, the subject is seropositive for human adenovirus prior to the administration. In some embodiments, the method(s) can further comprise administering a single dose of a second influenza pharmaceutical formulation about one year after administration of at least one dose of the previously administered influenza pharmaceutical formulation. In some such embodiments, the second influenza pharmaceutical formulation comprises antigens of a seasonal influenza that are the same or different as that comprised by the previously administered influenza pharmaceutical formulation. In some embodiments, the human subject is an adult.

Other embodiments are also contemplated herein as would be understood by those of ordinary skill in the art.

### EXAMPLES

The following examples are put forth so as to provide those of ordinary skill in the art with a complete disclosure and description of how to use the embodiments provided herein and are not intended to limit the scope of the disclosure nor are they intended to represent that the Examples below are all of the experiments or the only experiments performed. Efforts have been made to ensure accuracy with respect to numbers used (e.g. amounts, temperature, etc.) but some experimental errors and deviations should be accounted for. Unless indicated otherwise, parts are parts by volume, and temperature is in degrees Centigrade. It should be understood that variations in the methods as described can be made without changing the fundamental aspects that the Examples are meant to illustrate.

### Example 1: Preparation of Monovalent Influenza Pharmaceutical Formulation (NasoVAX)

The replication deficient adenoviral vector containing and expressing influenza virus hemagglutinin antigen codon optimized for the human subject was prepared following procedure detailed in [Lui J. et al.; A protocol for rapid generation of recombinant adenoviruses using the AdEasy system; Nat. Protoc. (2007) 2(5):1236-47].

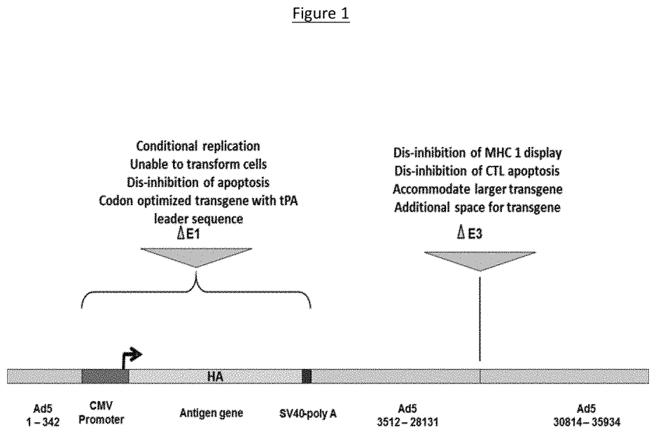

The present adenoviral vector is an E1/E3-deleted, replication deficient (RD)-Ad5 vector that expresses the protein of interest (e.g., Influenza HA) within respiratory epithelial cells. In the case of NasoVAX, the vector contains a genetic insert encoding the HA surface protein antigen from influenza type A or B. The recombinant Ad5 vector lacks the E1 region of the viral genome (nucleotides 343 to 3511), which renders the virus RD and incapable of producing infectious virus particles upon entry into a host cell. An additional deletion of nucleotides 28132 to 30813 in the E3 region of the vector removes genes that are involved in evading the host immune response and are dispensable for virus replication. An expression cassette consisting of a cytomegalovirus transcriptional enhancer/promoter to drive the expression of the HA gene, a bioengineered HA gene, and a Simian Virus 40 polyadenylation signal has been inserted in place of the E1 gene sequences. FIG. 1 provides a schematic diagram of the RD-Ad5 vector and identifies those sequences from the parent adenovirus genome that are retained in the vector.

In the present disclosure, the vector contained a genetic insert encoding the HA surface protein antigen from an A/California/04/2009(H1N1)-like strain of influenza (AdcoCA09.HA).

NasoVAX was manufactured by propagation of the RD-Ad5 vector in replication-permissive PER.C6 cells, followed by purification of the virus from the infected cell harvest, and the final product included the following excipients: Tris HCl (pH 7.4), histidine, sucrose, sodium chloride, magnesium chloride, polysorbate 80, ethylenediaminetetraacetic acid, and ethanol.

NasoVAX was supplied in single-use glass vials each containing a nominal volume of 0.7 mL of a sterile, frozen suspension of vaccine formulated to deliver the nominal doses of 1×10⁹ vp (batch number: 17142001), 1×10¹⁰ vp (batch number: 17143001), or 1×10¹¹ vp (batch number: 17144001).

### Example 2: Study Protocol: Single-Ascending-Dose Study of Immunogenicity of NasoVAX

Provided herein is a clinical study protocol wherein 60 healthy adults were randomized to an A/California 2009-based monovalent NasoVAX (present monovalent vaccine composition) formulation at doses of 10⁹, 10¹⁰, or 10¹¹ viral particles or saline placebo, all given as a 0.5 mL dose split approximately as 0.25 ml nasal spray in each nostril.

TABLE 1
Study Design

| | | Number of Subjects | |
|---|---|---|---|
| Cohort | Dose (vp) | NasoVAX | Placebo |
| 1 | 1 × 10⁹ | 15 | 5 |
| 2 | 1 × 10¹⁰ | 15 | 5 |
| 3 | 1 × 10¹¹ | 15 | 5 |

| | | |
|---|---|---|
| Study Total Target | 45 | 15 |

| | |
|---|---|
| Total | 60 |

The objectives of this study included: 1) To evaluate the humoral immune response to NasoVAX when administered by intranasal spray at a single dose of 1×10⁹, 1×10¹⁰, or 1×10¹¹ vp; 2) To evaluate the cellular immune response to NasoVAX when administered by intranasal spray at a single dose of 1×10⁹, 1×10¹⁰, or 1×10¹¹ vp; 3) To evaluate the mucosal immune response NasoVAX when administered by intranasal spray at a single dose of 1×10⁹, 1×10¹⁰, or 1×10¹¹ vp; and, 4) To evaluate the humoral immune response against non-represented influenza strains after NasoVAX administration.

Subjects were followed for safety, including solicited local and systemic side effects. Immune measures included hemagglutination inhibition (HAI) and neutralizing antibody (MN) at days one (1), 15, 29, 90 and 180, and γ-interferon ELISpot at day 1 and 8. A parallel cohort of 20 similar subjects were dosed with Fluzone® injectable influenza vaccine containing an A/California 2009 component and had assessments at the same timepoints.

This study was a Phase 2a, randomized, double-blind, placebo-controlled trial to evaluate the safety and immunogenicity of NasoVAX (monovalent Adco.CA.HA), i.e., NasoVAX, in healthy adults 18 to 49 years of age. Subjects were screened within 28 days of randomization (Day 1). Sixty subjects who met all inclusion and no exclusion criteria and provided written informed consent were enrolled into three (3) sequential cohorts of 20 subjects each defined by the vaccine dose (1×10⁹, 1×10¹⁰, and 1×10¹¹ vp). Within each cohort and its sentinel cohort, subjects were randomized in a 3:1 ratio to receive one (1) intranasal dose of NasoVAX or placebo on day one (Day 1).

A sentinel cohort of five (5) subjects from each cohort was dosed. Dosing of the remainder of each cohort proceeded after the last sentinel subject completed Day eight (8) if no events meeting stopping criteria had occurred. A serum sample was collected from each subject for HAI and microneutralization assays against influenza A/California/07/2009(H1N1) [a strain homologous to the one used for NasoVAX (monovalent AdcoCA.09.HA)] pre-dose on Day 1 and on Days 4, 8, 15, 29, 91, and 181; Ad5 antibody and HAI and microneutralization assays against non-represented influenza strains were also performed on the Day 1 and Day 29 samples. A whole blood sample was collected from each subject and processed to isolate PBMCs for evaluation of T-cell responses by ELISpot pre-dose on Day 1 and on Day 8.

A nasopharyngeal swab sample was collected from each subject at Screening (a time point prior to administration) and on Days 4, 8, 15, 29, and 91 to measure concentration of the Ad5 vector for assessment of vaccine vector shedding by quantitative polymerase chain reaction assay. Once a negative result was obtained for a given subject, later samples were not tested. ELISA for measurement of IgA was also performed on the swab samples from Screening and Day 29 for evaluation of mucosal immune response.

Immunogenicity:

Immunology analyses were conducted using the Per-protocol (PP) Population as the primary analysis population. Analyses based on the Intent-to-treat (ITT) Population were undertaken and presented only if >5% of subjects in any 1 dose group were excluded from the PP Population.

Analysis of covariance (ANCOVA) was used in the analysis of the antibody titer at each postbaseline visit, with log-transformed antibody titer as dependent variable, dose group as a factor, and Baseline log-transformed level as a covariate. Comparisons of postbaseline log-transformed antibody titer was conducted for each NasoVAX dose group against the placebo group and against the Fluzone® group, if applicable. From the ANCOVA analyses, least east squares (LS) means and 95% CI of the LS means of dose group, difference of LS means, and 95% CI of the difference in LS means were obtained. Back-transforming the difference of LS mean estimates and their 95% CIs to the original scale results in a ratio of the geometric means; these ratios were reported in the summary.

Categorical data derived from the assay data (e.g., SPR, SCR, responder rate) were tabulated by counts and percentages per dose group, as well as the 95% Clopper-Pearson exact CI of the percentage. In addition, comparisons of responders in each NasoVAX dose group against the placebo group and against the Fluzone® group, if applicable, were conducted using Fisher's exact test.

An analysis of median change from Baseline for Day 8 ELISpot SFU was added because median is the conventional parameter for descriptive analysis of raw ELISpot data and to account for differing Baseline group mean values.

Scatterplots of Baseline Ad5 antibody levels along the x-axis were presented with each of the following variables along the y-axis: HAI antibody titer at Day 29, ELISpot response at Day 8, and IgA titer at Day 29. The scatterplot analysis of effect of pre-existing Ad5 serum antibody levels on NasoVAX immunogenicity were uninformative, so post-hoc subgroup analysis by Baseline Ad5 serostatus (positive, defined as titer≥LLOQ, or negative) of Day 29 HAI assay GMR, Day 29 microneutralization assay GMR, median Day 8 change from Baseline in ELISpot SFU, and Day 29 IgA GMR was performed.

### Example 3: Combined Mucosal, Humoral and Cell-Mediated Immune Response Induced by Monovalent Influenza Pharmaceutical Formulation (NasoVax)

As described in Example 2, NasoVAX (monovalent AdcoCA09.HA) administered by intranasal spray at a single dose of 1×10⁹, 1×10¹⁰, or 1×10¹¹ vp elicited a combined humoral and mucosal immune responses and at a dose of 1×10¹¹ a combined immune response including a cellular response (e.g., T cells).

In certain embodiments provide herein is a monovalent influenza pharmaceutical formulation suitable for a single dose intranasal administration to a human subject, comprising: an effective amount of at least 10¹¹ viral particle (vp) of replication deficient adenovirus vector that contains and expresses influenza virus hemagglutinin antigen codon optimized for the human subject, wherein the effective amount induces a combined mucosal, humoral and T cell protective immune response; and, a pharmaceutically acceptable diluent or carrier.

In certain other embodiments provided herein is an influenza pharmaceutical formulation suitable for a single dose intranasal administration to a human subject, comprising: an effective amount of at least 10⁹ viral particles (vp) of replication deficient adenovirus vector that contains and expresses influenza virus hemagglutinin antigen codon optimized for the human subject, wherein the effective amount induces a combined mucosal and humoral protective immune response configured to provide seroprotection to the human subject of an HAI antibody titer ≥40 for at least 12 months against the influenza virus; and, a pharmaceutically acceptable diluent or carrier.

Also provided in certain embodiments is a method of inducing a combined mucosal, humoral and T cell protective immune response in a human subject against influenza virus whereby the human subject is seroprotected for at least 6 months. In embodiments, the method comprises administering intranasally to the human subject a single dose of any present influenza pharmaceutical formulation, wherein the administration induces serum antibodies, mucosal antibodies and T cells against influenza virus.

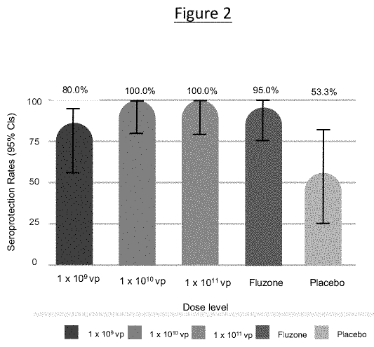

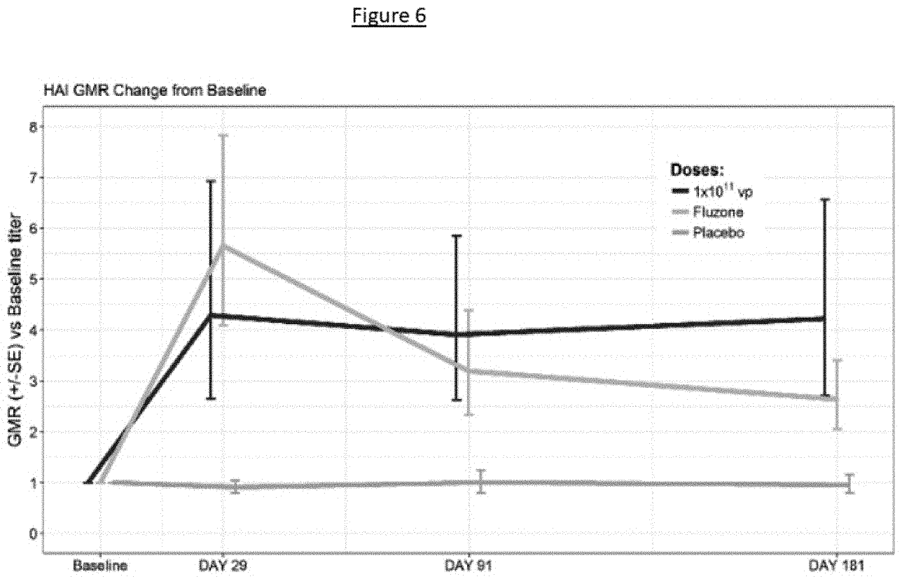

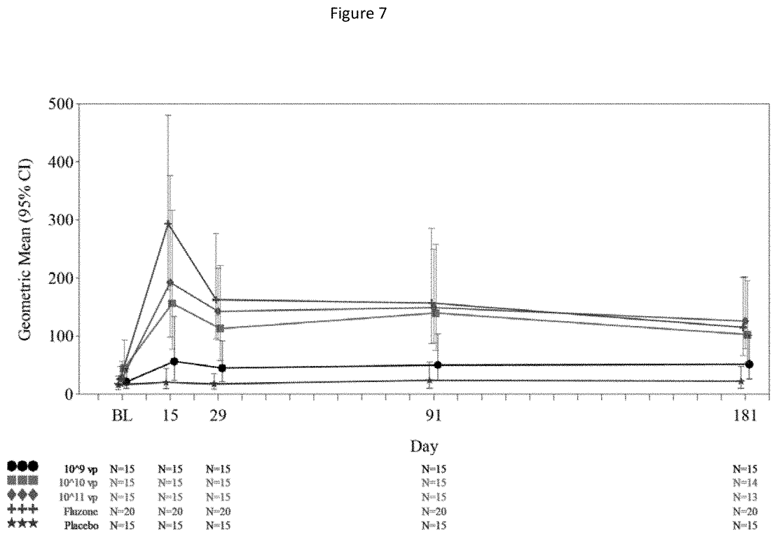

NasoVAX (monovalent AdcoCA09.HA) was administered by intranasal spray at a single dose of 1×10⁹, 1×10¹⁰, or 1×10¹¹ vp and found to elicit a humoral immune response to influenza A/California/07/2009(H1N1). All post-vaccination immunology results in the NasoVAX groups were superior to the placebo group from Day 15 through Day 181. For instance, a clear dose-effect was observed in the NasoVAX groups for HAI assay geometric mean titer (GMT), geometric mean ratio (GMR), seroconversion rate (SCR), and seroprotection rate (SPR); and for microneutralization GMT, GMR, and responder rates (2-fold and 4-fold). Except for HAI SCR in the overall group, all results in the NasoVAX 1×10¹¹ vp and overall groups at Day 29 were statistically significantly higher than those in the placebo group (P<0.05). For the microneutralization assay, Day 29 results in the 1×10¹¹ vp NasoVAX group were similar to those in the Fluzone® group; in the HAI assay, Day 29 results in the Fluzone® group were generally higher than in the NasoVAX groups. However, mean GMTs in the NasoVAX groups remained stable over time, while those in the Fluzone® group decrease substantially through Day 181. A post-hoc subgroup analysis of HAI and microneutralization GMTs by Baseline Ad5 serostatus showed some suppression of immune response to the hemagglutinin genetic insert in the Ad5 vector at the two lower doses, but this effect was apparently overcome in the highest dose group. See FIGS. 2-9.

NasoVAX elicited a HA-specific cellular immune response. Day 8 ELISpot results in the NasoVAX groups were higher than in the placebo group, and the difference was statistically significant in the 1×10¹¹ vp group (95% CI for LS GM SFUs ratio vs placebo: 3.5, 102.2; nonoverlapping 95% CIs for median change from Baseline; P=0.0028 for responder rate), and overall in the NasoVAX group (95% CI for LS GM SFUs ratio vs placebo: 1.6, 24.5; P=0.0351 for responder rate). Results in the 1×10⁹ vp and 1×10¹⁰ vp dose groups were slightly lower than in the Fluzone® group, while results in the 1×10¹¹ vp dose groups were substantially higher than in the Fluzone® group.

NasoVAX elicited HA-specific mucosal immunogenicity not seen in the subjects who received Fluzone®. GMRs at Day 29 showed mucosal response in all the NasoVAX groups and no response in the Fluzone® or placebo groups; the difference was statistically significant for the 1×10¹⁰ vp and 1×10¹¹ vp dose groups (95% CI: 1.6, 3.3 and 1.2, 2.9, respectively). Pre-existing Ad5 immunogenicity had no apparent effect on mucosal immunogenicity.

Humoral Immune Response

Humoral immune response (i.e., antibodies in the blood or serum) to the influenza HA protein as measured by HAI is a recognized correlate of protection for influenza (Clinical Data Needed to Support the Licensure of Seasonal Inactivated Influenza Vaccines, May 2007). Protection can be demonstrated via hemagglutination inhibition (HI) antibody response to a viral strain included in the vaccine, wherein GMT and SCR (defined as the percentage of subjects with either a pre-vaccination HI titer<1:10 and a post-vaccination HI titer≥1:40 or a pre-vaccination HI titer≥1:10 and a minimum four-fold rise in post-vaccination HI antibody titer) are measured to be in either a non-inferiority study or a placebo controlled study. For a non-inferiority study immunogenicity trial (HI antibody responses to a new vaccine as compared to an authorized influenza vaccine): 1) the upper bound of the two-sided 95% CI on the ratio of the GMTs (GMT of U.S. licensed vaccine divided by GMT of new vaccine) should not exceed 1.5; and, 2) the upper bound of the two-sided 95% CI on the difference between the SCRs (SCR for U.S. licensed vaccine less SCR for new vaccine) should not exceed 10 percentage points.

For a placebo-controlled immunogenicity trial, HI antibody response to the new vaccine may demonstrate protection when a percentage of subjects achieve an HI antibody titer≥1:40.

A blood sample was collected in a serum separation tube from each subject at day-28 to day-1, day 1, 2, 4, 8, 15, 29, 91 and 181 for evaluation of humoral response. HAI and microneutralization assays against A/California/07/2009(H1N1) were performed on all samples collected. After collection, samples were allowed to clot at room temperature for 30 minutes to 1 hour before centrifugation. The resulting serum component of the blood was transferred into aliquots in cryovials and stored at −80° C. for 24 to 48 hours before shipment on dry ice to the testing laboratories. Samples were stored at −80° C. before testing.

For the HAI assay, up to 9 samples were analyzed on each 96-well V-bottom microtiter plate (the test plate) along with quality (QC) controls on each plate consisting of positive (reference antiserum), negative, virus, and cell quality controls. A single QC plate was included in each assay and contained quality controls and a viral back titer (VBT) to confirm virus concentration on a 96-well V-bottom microtiter plate. Samples were treated with receptor destroying enzyme in accordance with the laboratory Standard Operation Procedure. Both test plates and QC plates were covered and incubated for 120 to 140 minutes at room temperature (15° C. to 30° C.). 50 μL of 1% horse red blood cells was then added to every well on all plates and incubated for 30 to 60 minutes at room temperature. Each well was then read for agglutination or non-agglutination and evaluated in accordance with the following criteria: 1) The titer was determined by the last well in the column that showed non-agglutination and was assigned the value of the reciprocal of that dilution. Serum dilutions were designated as 1:10 to 1:1280. A negative titer was assigned a titer of 5. Titers ≥1280 were assigned a titer of 1280 for use in GMT calculation; and, 2) The VBT on the QC plate had to be 4 HA units/25 μL±1 dilution for a valid assay. Samples were analyzed in triplicate for all assays.

For the microneutralization assay, up to nine (9) samples were analyzed on each 96-well flat-bottom microtiter plate (the test plate) along with QC on each plate consisting of a positive (reference antiserum), virus, and cell controls. At least three (3) QC plates were included in each assay and contained quality controls and a VBT to confirm virus concentration on a 96-well flat-bottom microtiter plate. Fifty microliters (50 μL) of diluted virus (diluted to target a VBT within the range of 1.17 to 9.38×TCID₅₀ [the amount of virus required to kill 50% of infected hosts or to produce a cytopathic effect in 50% of inoculated tissue culture cells]/50 μL) was added to all wells in all columns containing serum and the wells designated as the virus control; no virus was added to cell control wells. Both test plates and QC plates were incubated at 37° C.±2° C. with 0% to 5% CO₂) for 2 to 2.5 hours. One hundred microliters (100 μL) of MDCK cells (1.5×10⁵ cells/mL in diluent, 50% to 99% confluent) were added to all wells on every plate. Plates were incubated for 19 to 21 hours at 37° C.±2° C. with 5%±1% CO₂. Media was removed from all plates and each well washed with 100 μL Dulbecco's phosphate-buffered saline (DPBS, 1× concentration). DPBS was removed, and 100 μL of fixative was added to each well. Plates were covered and incubated at room temperature (15° C. to 30° C.) for 10 to 15 minutes. Fixative was removed, and plates were air-dried before washing 3 times with 200 μL/well of wash buffer. 100 mL of diluted primary antibody was added to each well, and plates were incubated for 1 to 1.25 hours at room temperature. Plates were washed four times with 200 mL/well of wash buffer, and 100 mL of diluted secondary antibody was added to each well. Plates were incubated for 1 to 1.25 hours at room temperature. Plates were washed four times with 200 mL/well of wash buffer, and 100 mL fresh substrate was added (in low light) to each well followed by incubation for 15 to 25 minutes in low light at room temperature. Stop solution (100 mL) was immediately added to each well, and absorbance was read at 490 to 495 nm. The resulting data were evaluated in accordance with the following criteria: 1) the plate cut-off value for each plate was calculated according to the following equation: (average of virus control wells—average of cell control wells)/2; and, 2) serum samples with an absorbance value below the plate cut-off were positive for neutralizing antibody, whereas those above the plate cut-off were negative for neutralizing antibody. VBT values with an absorbance value above the plate cut-off were positive for virus replication. VBT values with an absorbance value below the plate cut-off were negative for virus replication. The titer was calculated as the reciprocal of the highest dilution scored as a positive. A negative titer (all absorbance values above the plate cut-off) was assigned a value of 5. Dilutions were designated 1:10 to 1:1280. Titers >1280 were assigned a titer of 1280 for use in GMT calculations but were reported as >1280 in the data tables. Exceptions included: absorbance value that crossed over the plate cut-off value (value fell below the cut-off, rose above it (noted as ‘flag’), then fell back below) had the titer assigned for the final absorbance value that fell below the plate cut-off value unless there was a ≥2-fold difference between the initial fall in absorbance and when it rose again above it; samples with more than one flag were not accepted unless a mechanical issue was noted on data evaluation or the final absorbance value was more than two dilutions from the last flag; values that crossed over the plate cut-off in the VBT were reported as the titer prior to the cross; and, when evaluating absorbance values on all plates, data trending was evaluated down and across plates to look for indications of mechanical issues (e.g., partial or complete clogged tip that could result in absorbance values higher or lower than expected). These values were taken into consideration when evaluating and assigning the final titer of the sample.

Test plates and the assay were acceptable if the following criteria were met: 1) Positive QC values on individual test plates were within (±) 1 dilution of the calculated median value for the assay, or the individual plate was rejected; 2) The VBT median value determined for all accepted QC plates for an assay was in the range of 1.17 to 9.38, or the assay was rejected; and, 3) At least 5 of the 9 serum samples on a test plate match at least 1 of their respective replicates, or all 9 test samples were reanalyzed. Samples were analyzed in triplicate for all assays.

All post-vaccination results in the NasoVAX groups were higher than in the placebo group from Day 15 through Day 181. A clear dose effect was seen in the NasoVAX groups for all the endpoints measured. Except for HAI SCR in the overall group, all humoral immunogenicity results in the NasoVAX 1×10¹¹ vp and overall groups at Day 29 were statistically significantly higher than those in the placebo group. See FIGS. 2, 3, 6 and 7.

TABLE 1
Humoral Immune Response to NasoVAX at Day 29 by Dose Group

| | NasoVAX | | | | | |
|---|---|---|---|---|---|---|
| | 1 × 10⁹ vp | 1 × 10¹⁰ vp | 1 × 10¹¹ vp | Overall | Placebo | Fluzone |
| Statistic | N = 15 | N = 15 | N = 15 | N = 45 | N = 15 | N = 20 |

HAI Assay

| | | | | | | |
|---|---|---|---|---|---|---|
| LS GMT (95% CI)^{a} | 87.2 | 136.1 | 164.0 | 124.8 | 31.3 | 277.7 |
| | (52.7, 144.3) | (81.7, 226.6) | (99.0, 271.6) | (92.3, 167.0) | (18.9, 52.0) | (179.4, 429.9) |
| LS GMT ratio vs | 2.8 | 4.3 | 5.2 | 4.0 | | |
| placebo (95% CI)^{a} | (1.4, 5.7) | (2.1, 9.0) | (2.6, 10.7) | (2.2, 7.2) | | |
| LS GMT ratio vs | 0.3 | 0.5 | 0.6 | 0.4 | | |
| Fluzone (95% CI)^{a} | (0.2, 0.6) | (0.3, 1.0) | (0.3, 1.2) | (0.3, 0.8) | | |
| GMR (95% CI) | 2.1 | 2.2 | 4.3 | 2.7 | 0.9 | 5.7 |
| | (1.1, 4.2) | (1.2, 4.3) | (1.5, 12.0) | (1.8, 4.2) | (0.7, 1.2) | (2.9, 11.2) |
| SCR^{b} (95% CI) | 13.3% | 26.7% | 33.3% | 24.4% | 0.0% | 50.0% |
| | (1.7%, 40.5%) | (7.8%, 55.1%) | (11.8%, 61.6%) | (12.9%, 39.5%) | (0.0%, 21.8%) | (27.2%, 72.8%) |
| P value vs placebo^{c} | 0.4828 | 0.0996 | 0.0421 | 0.0505 | | |
| P value vs Fluzone^{c} | 0.0340 | 0.2958 | 0.4916 | 0.0506 | | |
| SPR^{d} (95% CI) | 80.0% | 100.0% | 100.0% | 93.3% | 53.3% | 95.0% |
| | (51.9%, 95.7%) | (78.2%, 100.0%) | (78.2%, 100.0%) | (81.8%, 98.6%) | (26.6%, 78.7%) | (75.1%, 99.9%) |
| P value vs placebo^{c} | 0.2451 | 0.0063 | 0.0063 | 0.0013 | | |
| P value vs Fluzone^{c} | 0.2924 | 1.000 | 1.000 | 1.000 | | |

Microneutralization Assay

| | | | | | | |
|---|---|---|---|---|---|---|
| GMT (95% CI) | 44.9 | 113.1 | 142.5 | 89.8 | 17.8 | 162.8 |
| | (21.8, 92.3) | (58.0, 220.8) | (93.6, 217.1) | (62.6, 128.8) | (9.1, 35.0) | (95.8, 276.6) |
| GMR (95% CI) | 2.1 | 2.5 | 5.2 | 3.0 | 1.1 | 6.2 |
| | (!.0, 4.2) | (1.3, 4.8) | (2.2, 12.0) | (2.0, 4.5) | (1.0, 1.2) | (2.8, 13.8) |
| Responder rate/2- | 40.0% | 46.7% | 73.3% | 53.3% | 0.0% | 70.0% |
| fold rise (95% CI) | (16.3%, 67.7%) | (21.3%, 73.4%) | (44.9%, 92.2%) | (37.9%, 68.3%) | (0.0%, 21.8%) | (45.7%, 88.1%) |
| P value vs placebo^{c} | 0.0169 | 0.0063 | <0.0001 | 0.0001 | | |
| P value vs Fluzone^{c} | 0.0966 | 0.1871 | 1.0000 | 0.2785 | | |
| Responder rate/4- | 13.3% | 26.7% | 53.3% | 31.1% | 0.0% | 50.0% |
| fold rise (95% CI) | (1.7%, 40.5%) | (7.8%, 55.1%) | (26.6%, 78.7%) | (17.6%, 44.6%) | (0.0%, 21.8%) | (27.2%, 72.8%) |
| P value vs placebo^{c} | 0.4828 | 0.0996 | 0.0022 | 0.0130 | | |
| P value vs Fluzone^{c} | 0.0340 | 0.2958 | 1.0000 | 0.1716 | | |

Bold values are statistically higher than values in placebo group.
Abbreviations: CI = confidence interval; GMT = geometric mean titer; GMR = geometric mean ratio; HAI = hemagglutination inhibition; LS = least squares; SCR = reconversion rate; SPR = seroprotection rate; vp = viral particles.
^{a}The analysis of covariance uses log-transformed level as dependent variable, dose group as a factor, and Baseline log-transformed analysis as a covariate. Differences of LS mean estimates and 95% CIs were back-transformed to the original scale, resulting in a ratio of the geometric means.
^{b}The percentage of subjects with a HAI titer ≥ 1:40
^{c}From Fisher's exact test
^{d}The percentage of subjects with either a Baseline HAI titer < 1:10 and a postvaccination titer ≥ 1:40 (which is 4 times the assay lower limit of quantitation), or a Baseline HAI titer ≥ 1:10 and a 4-fold increase in postvaccination HAI titer relative to Baseline

Cellular Immune Response

A blood sample was collected in a heparinized tube from each subject at day-28 to day-1, day 1, 2, 4, 8, 15, 29, 91 and 181 for evaluation of HA-specific T-cell response by ELISpot.

PBMCs were isolated by standard Ficoll-Hypaque gradient density technique with centrifugation steps adjusted to obtain optimal yield and viability with site equipment. PBMCs were cryopreserved at a density of 10⁷ cells/mL and stored at −80° C. before shipment on dry ice to the testing laboratory. Samples were stored on liquid nitrogen before testing.

After thawing, PBMCs were added at a concentration of 0.2×10⁶ cells per well to a 96-well polyvinyl difluoride membrane ELISpot plate coated with the capture interferon gamma (IFN-γ) antibody. PBMCs were stimulated for 18 hours in the presence of 7 pooled HA-derived peptides, positive and negative controls. HA-derived peptide pools (19 to 20 peptides each) were prepared from 139 short peptides (14 or 15 amino-acid long, 11 amino-acid overlap) derived from the HA sequence of the A/California/04/2009(H1N1) strain. Positive controls used a CEF (cytomegalovirus, Epstein Barr virus and influenza virus) peptide pool and phytohemagglutinin. Negative controls were based on irrelevant human myelin oligodendrocyte glycoprotein peptide pool and media alone. All conditions were tested in triplicate except media alone, which was tested in sextuplicate. After revelation of the IFN-γ ELISpot plate, the number of spots was counted with an automated ELISpot counter and expressed as SFU/10⁶ PBMCs.

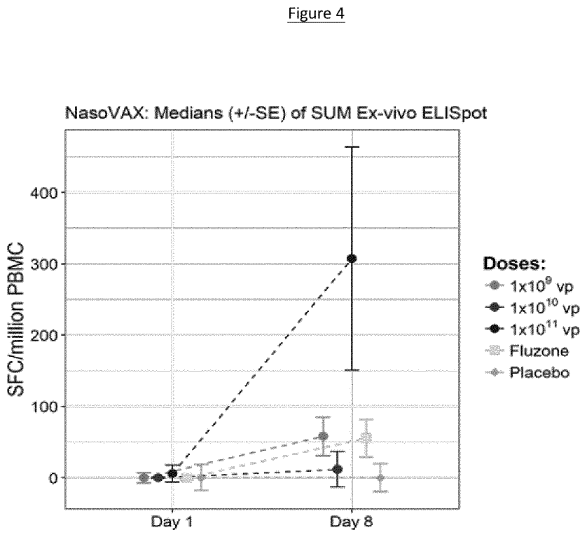

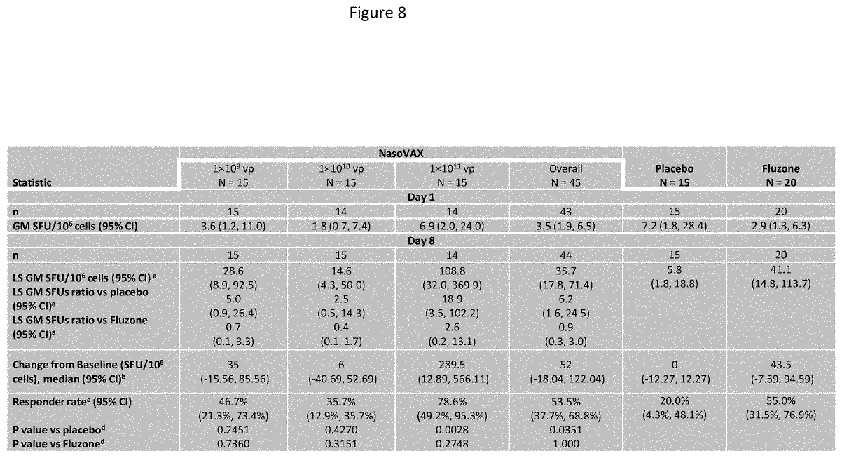

FIGS. 4 and 8 summarize the cellular immune response (SFUs from ELISpot) at Day 8. Baseline geometric mean SFU/10⁶ cells varied widely among the dose groups and, therefore, a post-hoc analysis of change from Baseline was added. Post-vaccination results in the NasoVAX groups were higher than in the placebo group, and the difference was statistically significant in the 1×10¹¹ vp, overall NasoVAX groups for LS GM SFUs and responder rate, and in the 1×10¹¹ vp group for median change from Baseline.

Mucosal Immune Response

Nasopharyngeal swab samples collected at Screening and Day 29 and used for evaluation of mucosal response by anti-HA IgA ELISA. After nasopharyngeal swab sample collection, the swab was frozen at −80° C. before being shipped on dry ice to the testing laboratory and stored at −20° C. before use.

Influenza A/California/04/2009(H1N1) HA protein was adsorbed onto the surface of microtiter wells. Nasal swab extract (i.e., sample) was incubated with HA (i.e., allowed to bind to HA), and a biotinylated detection antibody added to the wells to detect the captured IgA (i.e., mucosal IgA). The anti-HA IgA titer was determined using a cut-off absorbance-based method. The total IgA per sample was also quantitated using the Human IgA ELISA Quantitation Set, E80-102 (Bethyl Laboratories; Montgomery, Tex.). The results were reported as a ratio of HA-specific IgA (U/mL) to total Ig A (μg/ml) to allow for direct comparison between different nasal swab samples. Results were transferred electronically into the study database.

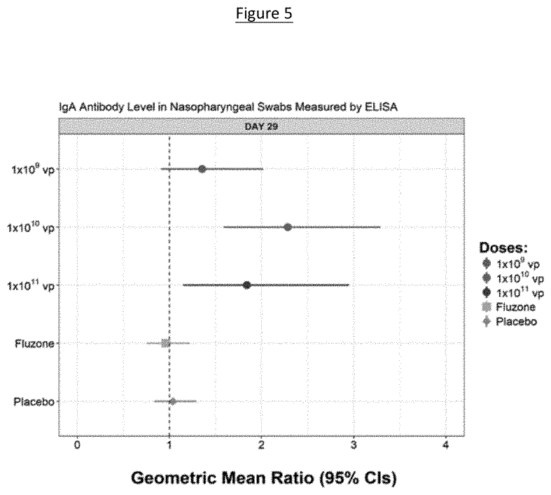

Table 2 and FIG. 5 summarize mucosal immune response to vaccine strain HA protein (IgA by ELISA) at Day 29. Unlike for humoral antibodies (i.e., antibodies present in blood), mucosal IgA GMTs were similar across all groups at Baseline (range: 1.8 to 3.0). GMRs at Day 29 showed no mucosal response in the placebo group and mucosal response in all the NasoVAX groups; the difference to placebo was statistically significant for the 1×10¹⁰ vp and the 1×10¹¹ vp groups dose groups.

TABLE 2
Mucosal Immune Response to NasoVAX at Day 29 by Dose Group

| | NasoVAX | | | | | |
|---|---|---|---|---|---|---|
| | 1 × 10⁹ vp | 1 × 10¹⁰ vp | 1 × 10¹¹ vp | Overall | Placebo | Fluzone ® |
| Statistic | N = 15 | N = 15 | N = 15 | N = 45 | N = 15 | N = 20 |
| GMT (95% CI) | 3.3 | 5.6 | 5.4 | 4.6 | 2.4 | 1.8 |
| | (2.1, 5.1) | (3.8, 8.3) | (3.2, 9.4) | (3.6, 6.0) | (2.0, 2.9) | (1.2, 2.3) |
| GMR (95% CI) | 1.4 | 2.3 | 1.8 | 1.8 | 1.0 | 1.0 |
| | (0.9, 2.0) | (1.6, 3.3) | (1.2, 2.9) | (1.1, 1.2) | (0.8, 1.3) | (0.8, 1.2) |

Abbreviations: CI = confidence interval; GMT = geometric mean titer; GMR = geometric mean ratio; vp = viral particles.

### DISCUSSION

The humoral, cellular, and mucosal immunogenicity elicited by NasoVAX (monovalent AdcoCA09.HA) administered by intranasal spray at a single dose of 1×10⁹, 1×10¹⁰, or 1×10¹¹ vp showed the potential to address some of the shortcomings in current influenza vaccine options noted in the recent NIAID review (Erbelding E J, et al. (2018)), specifically inadequate durability of immune response, poor cellular response, and lack of tissue-resident (i.e., mucosal) immunity. In this study, the humoral immune response (including that measured by the HAI assay, the standard correlate of protection) remained stable in the NasoVAX groups through six (6) months post-dose, while response in the Fluzone® group decreased substantially over that time period.

Cellular and mucosal immunogenicity may play a role in reduction of influenza duration, severity, and transmissibility (Gould, et al. Nasal IgA provides protection against human influenza challenge in volunteers with low serum influenza antibody titre. Front Microbiol. 2017; 8:900; McMichael, et al. Cytotoxic T-cell immunity to influenza. N Engl J Med. 1983; 309:13-7; Seibert, et al. Recombinant IgA is sufficient to prevent influenza virus transmission in guinea pigs. J Virol. 2013; 87:7793-804; Wilkinson, et al. Preexisting influenza specific CD4+ T cells correlate with disease protection against influenza challenge in humans. Nat Med. 2012; 18:274-80), characteristics that are particularly important in the setting of pandemic influenza. The mucosal immune response observed in these studies has the potential to provide an effect additive to the humoral and cellular response pathways by blocking influenza at the site of infection. In this study, the cellular response elicited by NasoVAX was superior to that from Fluzone®, and mucosal immune response was elicited by NasoVAX but not by Fluzone.

The immunogenicity results in this study clearly indicate that the 1×10¹¹ vp dose elicits strong humoral, cellular, and mucosal immunogenicity. The effect of the 1×10¹⁰ vp dose was less clear, possibly because of the unusually high Baseline HAI and microneutralization titers in this group.

### Example 4: Comparison of Induced Immune Response by Monovalent Influenza Pharmaceutical Formulation (NasoVAX) and Fluzone®

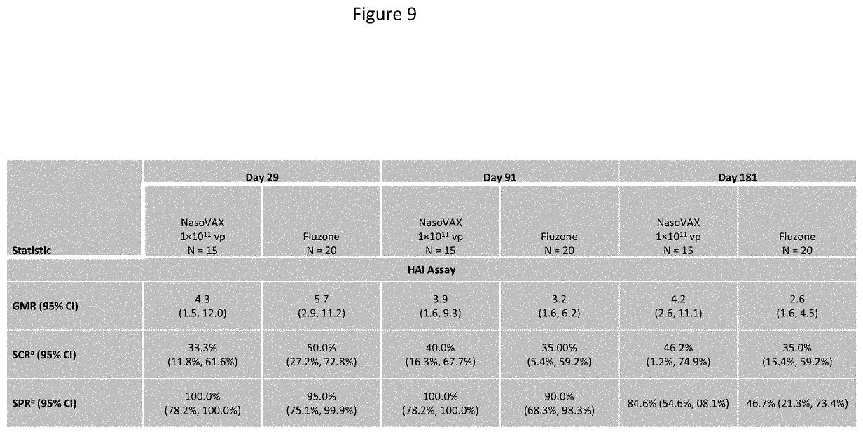

The immunogenicity, including the ability to induce a humoral, cellular and mucosal immune response, was evaluated for the present monovalent influenza pharmaceutical formulation and compared to Fluzone®. Fluzone® is a high-dose quadrivalent influenza vaccine administered via injection for influenza A and influenza B subtypes present in the vaccine. HAI antibodies were measured at day 29, 91 and 181 and results reported for a HAI assay, seroconversion rate and seroprotection. See FIG. 9.

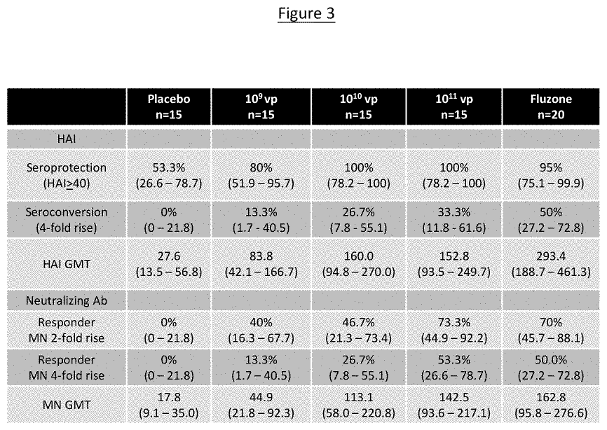

For the microneutralization (MN) assay, as shown in FIG. 3, Day 29 results in the 1×10¹¹ vp NasoVAX group were similar to those in the Fluzone® group; in the HAI assay, Day 29 results in the Fluzone group were generally higher than in the NasoVAX groups. However, mean GMTs by both assays and HAI GMR, SCR, and SPR values in the NasoVAX groups remained stable over time, while those in the Fluzone® group decreased substantially through Day 181. See FIG. 9.

NasoVAX (monovalent AdcoCA09.HA) administered by intranasal spray at a single dose of 1×10⁹, 1×10¹⁰, or 1×10¹¹ vp elicited a humoral immune response to influenza A/California/07/2009(H1N1). All postvaccination results in the NasoVAX groups were higher than in the placebo group from Day 15 through Day 181. A clear dose effect was seen in the NasoVAX groups for HAI assay GMT, GMR, SCR, and SPR and for microneutralization GMT, GMR, and responder rates (2-fold and 4-fold). Except for HAI SCR in the overall group, all results in the NasoVAX 1×10¹¹ vp and overall groups at Day 29 were statistically significantly higher than those in the placebo group (P<0.05). For the microneutralization assay, Day 29 results in the 1×10¹¹ vp NasoVAX group were similar to those in the Fluzone® group; in the HAI assay, Day 29 results in the Fluzone® group were generally higher than in the NasoVAX groups. However, mean GMTs in the NasoVAX groups remained stable over time, while those in the Fluzone® group decrease substantially through Day 181.

In the cellular immunogenicity assay (ELISpot), results in the 1×10⁹ vp and 1×10¹⁰ vp dose groups were slightly lower than in the Fluzone® group, while results in the 1×10¹¹ vp dose group were substantially higher than in the Fluzone® group. See FIG. 8.

NasoVAX elicited a strain-specific cellular immune response. Day 8 ELISpot results in the NasoVAX groups were higher than in the placebo group, and the difference was statistically significant in the 1×10¹¹ vp group (95% CI for LS GM SFUs ratio vs placebo: 3.5, 102.2; nonoverlapping 95% CIs for median change from Baseline; P=0.0028 for responder rate) and overall NasoVAX group (95% CI for LS GM SFUs ratio vs placebo: 1.6, 24.5; P=0.0351 for responder rate). Results in the 1×10⁹ vp and 1×10¹⁰ vp dose groups were slightly lower than in the Fluzone® group while results in the 1×10¹¹ vp dose groups were substantially higher than in the Fluzone® group. See FIG. 8.

In the mucosal immunogenicity assay (IgA ELISA in nasopharyngeal swabs samples), GMRs at Day 29 showed mucosal response in all the NasoVAX groups and no mucosal response in the Fluzone® group; the difference was statistically significant for the 1×10¹⁰ vp and 1×10¹¹ vp dose group (95% CIs for GMT and GMR did not overlap with 95% CI for the Fluzone group). See FIG. 5.

NasoVAX elicited strain-specific mucosal immunogenicity not seen in the subjects who received Fluzone®. GMRs at Day 29 showed mucosal response in all the NasoVAX groups and no response in the Fluzone® or placebo groups; the difference was statistically significant for the 1×10¹⁰ vp dose and 1×10¹¹ vp dose groups (95% CI: 1.6, 3.3 and 1.2, 2.9, respectively).

### Example 5: Durability: Seroprotection for 13 Months Induced by Monovalent Influenza Pharmaceutical Formulation (NasoVAX)

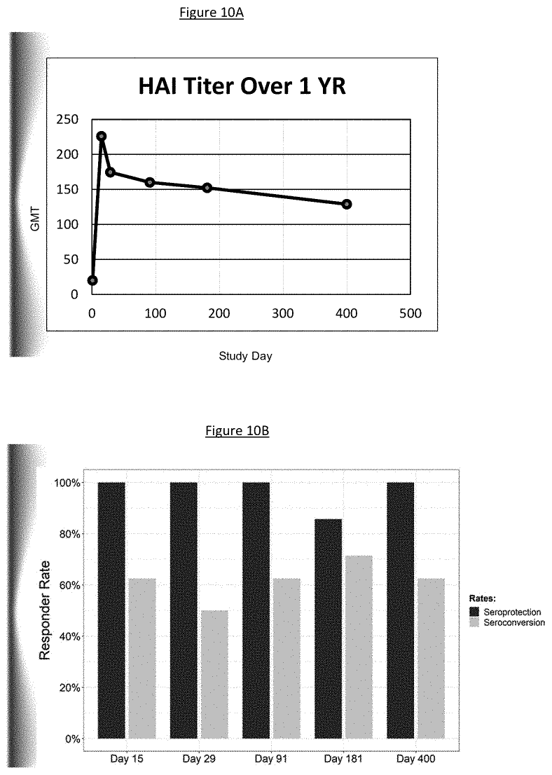

The above study disclosed in Example 2 and 3 was extended an additional six (6) months wherein a blood sample was collected in a serum separation tube from each available subject between day 403 and day 433 for evaluation of humoral response. In total eight of the 15 subjects at the 1×10¹¹ vp dose group returned for evaluation after one year. HAI and microneutralization assays against A/California/07/2009(H1N1) were performed on all eight samples collected. All subjects that participated in the extension study demonstrated seroprotection at least 13 months post administration with the present monovalent influenza vaccine. See Table 3 and FIG. 10.

TABLE 3
Seroprotection 13 to 14 months post vaccination

| | Vaccine | Blood | | |
|---|---|---|---|---|
| | Administered | collection | | Seroprotected |
| SUBJID | Date | at Day 365 | Days | HAI titer |
| 1 | Nov. 28, 2017 | Jan. 21, 2019 | 419 | 226 |
| 2 | Dec. 12, 2017 | Jan. 21, 2019 | 405 | 56.6 |
| 3 | Dec. 13, 2017 | Jan. 23, 2019 | 406 | 320 |
| 4 | Dec. 13, 2017 | Jan. 22, 2019 | 405 | 160 |
| 5 | Dec. 14, 2017 | Feb. 18, 2019 | 431 | 320 |
| 6 | Dec. 14, 2017 | Jan. 21, 2019 | 403 | 320 |
| 7 | Dec. 13, 2017 | Jan. 22, 2019 | 405 | 226 |
| 8 | Dec. 12, 2017 | Feb. 18, 2019 | 433 | 80 |

### Example 6. NasoVAX Shedding and Anti-NasoVAX Vector Antibodies

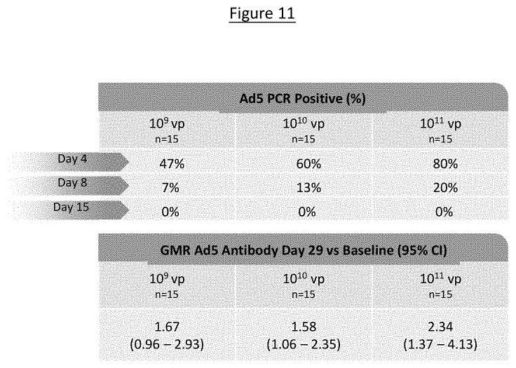

NasoVAX was administered to human subjects as a single intranasal dose of 10⁹ vp, 10¹⁰ vp, or 10¹¹ vp. At four, eight and 15 days post-dose, nasopharyngeal swab samples were collected from each subject and the concentration of the Ad5 vector shed at each time point quantified by polymerase chain reaction (PCR) assay. As shown in FIG. 11, dose-dependent shedding of administered NasoVAX vector dose was detected until day 8 post-dose and was not detected at day 15. No replication-competent virus was detected.

An adenovirus microneutralization (MN) assay was carried out to determine anti-adenovirus antibodies in subjects. Dilutions of human sera were mixed with a consistent quantity of a replication deficient Ad5 vector that expresses green fluorescent protein (GFP) from a human cytomegalovirus (CMV) promoter (termed Ad5.CMV-GFP) and incubated to allow potential neutralization to occur. These mixtures were then inoculated onto VERO E6 cells in a 96-well plate and incubated for approximately three days. During this incubation, Ad5.CMV-GFP that is not neutralized infects the cells and produce GFP, which was used as a readout for the assay. The intensity of the GFP fluorescence was compared between test samples and controls that are either inoculated with virus only or no virus to determine the level of neutralization that occurred. The reportable value for this assay is the MN50 value, corresponding to the reciprocal of the serum dilution that results in neutralization of 50% of the input Ad5.CMV-GFP. FIG. 11 also illustrates the GMR of antibodies against the adenovirus vector component of NasoVAX (Ad5) following administration of a single intranasal dose of 10⁹ vp, 10¹⁰ vp, or 10¹¹ vp of NasoVAX. As shown therein, administration of the highest dose (10¹¹ vp) surprisingly only resulted in about a 2.3-fold induction of anti-Ad5 vector antibodies in subjects as compared to control. This is an important finding as it indicates the intranasal route of administration can be used for repeated dosing of NasoVAX, or potentially other Ad5-based vectors.

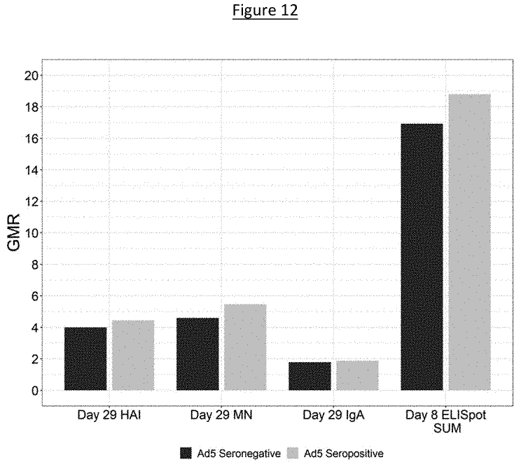

FIG. 12 shows the effect of pre-existing anti-Ad5 immunity on Ad5 serostatus following administration of a single intranasal dose (10¹¹ vp) of NasoVAX to subjects. As shown therein, pre-existing anti-Ad5 immunity (“Ad5 Seropositive” (median titer being 22-fold above the lower limit of quantitation (LLOQ)) had little effect on humoral (HAI), microneutralization (MN), mucosal (IgA), or cellular (ELISpot) anti-Ad5 immunity following administration of the intranasal dose of NasoVAX. This is another important finding as it indicates that NasoVAX can be administered intranasally even to subject with pre-existing immunity to Ad5.

### Example 7. NasoVAX Stability at Room Temperature

This example describes the long-term stability of NasoVAX in a liquid formulation at room temperature. Long-term stability at room temperature is desire feature of vaccines that can be used in situations in which refrigeration or other means for stabilizing a formulation may not be available. This would be important in epidemic or pandemic situations during which vaccines need to be shipped to remote areas that may lack the equipment to maintain formulations at a cooler temperature. As shown in Tables 4 and 5 below, low dose (2×10⁹ vp/mL dose) and high dose (2×10¹¹ vp/mL dose) formulations, respectively, were prepared and maintained at 25 Cin glass vials for one, three and six months. Viability of the NasoVAX vectors was determined using the Adenovirus Fluorescent Focus Unit (FFU) assay. Briefly, the FFU assay is carried out by infecting cell monolayers with the appropriate NasoVAX dilution and incubated for 24-48 hours. The cells were then washed, inspected, fixed (e.g., ice-cold 90% methanol for four minutes), and washed again. Anti-Ad5 antibody was then added at various dilutions (antibody omitted in control samples), followed by a detection agent (e.g., NCL-Adeno (Novocastra, Newcastle, UK)) under appropriate conditions (e.g., ten minutes at room temperature with shaking). The cells are then washed, and the total number of infectious particles determined (e.g., by digital light scattering (DLS)). As shown in Tables 4 and 5, the low-dose and high-dose NasoVAX formulations were stable for at least three months at room temperature.

TABLE 4
Stability Data for NasoVAX (2 × 10⁹ vp/mL dose)

| | Stability Time Point | | | |
|---|---|---|---|---|
| Analysis | T = 0M | T = 1M | T = 3M | T = 6M |
| Appearance | Liquid, | Liquid, | Liquid, | Liquid, |
| | Colorless; | Colorless, | Colorless; | Colorless; |
| | Translucent; | Translucent; | Clear; | Transparent; |
| | No visible | No visible | No visible | No visible |
| | particulate | particulate | particulate | particulate |
| | matter | matter | matter | observed |
| | observed | observed | observed | |
| pH | 7.5 | 7.5 | 7.7 | 7.5 |
| vp by HPLC | 1.2 × 10⁹ | 1.1 × 10⁹ | 0.9 × 10⁹ | 1.2 × 10⁹ |
| | vp/mL | vp/mL | vp/mL | vp/mL |
| Adenovirus | 1.1 × 10⁸ | 2.3 × 10⁸ | 0.7 × 10⁸ | 0.1 × 10⁸ |
| Fluorescent | FFU/mL | FFU/mL | FFU/mL | FFU/mL |
| Focus Unit | | | | |
| (FFU) Assay | | | | |
| % Infectious | 9% | 21% | 8% | 0.4% |
| Particles | | | | |
| Aggregation | 66.7 nm | 139.7 nm | 91.9 nm | 107.5 nm |
| by DLS | | (23% PD) | (14% PD) | (8% PD) |

TABLE 5
Stability Data for Naso VAX (2 × 10¹¹ vp/mL dose)

| | Stability Time Point | | | |
|---|---|---|---|---|
| Analysis | T = 0M | T = 1M | T = 3M | T = 6M |
| Appearance | Liquid, | Liquid, | Liquid, | Liquid, |
| | Colorless; | Colorless; | Colorless; | Colorless; |
| | Translucent; | Translucent; | Translucent; | Translucent; |
| | No visible | No visible | No visible | No visible |
| | particulate | particulate | particulate matter | particulate |
| | matter | matter | observed | observed |
| | observed | observed | | |
| pH | 7.6 | 7.5 | 7.5 | 7.6 |
| vp by HPLC | 1.3 × 10¹¹ | 1.0 × 10¹¹ | 0.4 × 10¹¹ | 1.2 × 10¹¹ |
| | vp/mL | vp/mL | vp/mL | vp/mL |
| Adenovirus | 0.9 × 10¹⁰ | 0.9 × 10¹⁰ | 0.5 × 10¹⁰ | 0.1 × 10¹⁰ |
| Fluorescent | FFU/mL | FFU/mL | FFU/mL | FFU/mL |
| Focus Unit | | | | |
| (FFU) Assay | | | | |
| % Infectious | 7% | 9% | 12% | 0.5% |
| Particles | | | | |
| Aggregation | 122 nm | 118.2 nm | 116.9 nm | 115.5 nm |
| by DLS | | (19% PD) | (13% PD) | (14% PD) |

While certain embodiments have been described in terms of the preferred embodiments, it is understood that variations and modifications will occur to those skilled in the art. Therefore, it is intended that the appended claims cover all such equivalent variations that come within the scope of the following claims.

1. An influenza pharmaceutical formulation suitable for a single dose intranasal administration to a human subject, comprising:
an effective amount of at least 10¹¹ viral particles (vp) of replication deficient adenovirus vector that contains and expresses influenza virus hemagglutinin antigen (HA) codon optimized for the human subject, wherein the effective amount induces a combined mucosal, humoral and T cell immune response; and,
a pharmaceutically acceptable diluent or carrier.

2. The formulation of claim 1, wherein:
wherein the mucosal immune response is determined by anti-hemagglutinin (HA) IgA ELISA, the humoral immune response is determined by hemagglutination inhibition assay (HAI) titer and/or the presence of neutralizing antibody as determined using a microneutralization assay, optionally as measured using one or more of the geometric mean titer (GMT), geometric mean ratio (GMR), seroconversion rate (SCR), seropositivity rate (SPR); and/or,
the T cell immune response is determined by using γ-interferon ELISpot.

3. The formulation of claim 1 wherein the combined mucosal, humoral, and T cell immune response is protective.

4. The formulation of claim 3, wherein the formulation is configured to provide seroprotection to the human subject as determined by the subject having an HAI antibody titer ≥40 for at least 12 months against the influenza virus.

5. A pharmaceutical formulation suitable for a single dose intranasal administration to a human subject, comprising:
an effective amount of at least 10⁹ viral particles (vp) of replication deficient adenovirus vector that contains and expresses influenza virus hemagglutinin antigen (HA) codon optimized for the human subject, wherein the effective amount induces a combined mucosal and humoral protective immune response configured to provide seroprotection to the human subject as determined by the subject having an HAI antibody titer ≥40 for at least 12 months against the influenza virus; and,
a pharmaceutically acceptable diluent or carrier.

6. The formulation of claim 5, wherein the effective amount is at least 10¹⁰ viral particles (vp), or wherein the effective amount is at least 10¹¹ viral particles (vp) and further induces a T cell response.

7-9. (canceled)

10. The formulation of claim 1, wherein the HA antigen is from an Influenza A virus, Influenza A virus subtype H1N1, or Influenza A virus subtype H3N2.

11. (canceled)

12. (canceled)

13. The formulation of claim 5 wherein the formulation is frozen or wherein the formulation is stable at ambient temperature for at up to about three months.

14-18. (canceled)

19. The formulation of claim 5 wherein the formulation is within a container selected from the group consisting of a glass vial, nasal sprayer, droplet dispenser, aerosolizer, and atomizer.

20. (canceled)

21. A container comprising a formulation of claim 5, wherein the container has contained the formulation for up to about three months at ambient temperature.

22-25. (canceled)

26. The container of claim 21, wherein the container is a single-use container or comprises multiple doses; and/or is configured for intranasal administration of the formulation.

27. (canceled)

28. (canceled)

29. (canceled)

30. A method of inducing a combined mucosal, humoral and T cell immune response in a human subject against influenza virus comprising:
administering intranasally to a human subject a single dose of the influenza pharmaceutical formulation of 1,
wherein the administration induces a combined mucosal, humoral and T cell immune response against influenza virus.

31. The method of claim 30 wherein:
the mucosal immune response is determined by anti-hemagglutinin (HA) IgA ELISA, the humoral immune response is determined by hemagglutination inhibition assay (HAI) titer and/or presence of neutralizing antibody as determined using a microneutralization assay, optionally as measured using one or more of the geometric mean titer (GMT), geometric mean ratio (GMR), seroconversion rate (SCR), seropositivity rate (SPR); and/or,
the T cell immune response is determined by using γ-interferon ELISpot.

32. (canceled)

33. The method of claim 30 wherein the human subject is seroprotected from infection by influenza virus for at least 12 months, at least 13 months, or at least 14 months after said administration.

34-35. (canceled)

36. The method of claim 30, wherein the influenza virus is a seasonal influenza virus.

37. (canceled)

38. The method of claim 30, wherein administration induces an HAI antibody titer of at least 50 for at least 12 months post administration.

39. (canceled)

40. (canceled)

41. The method of claim 30 wherein administration of the formulation does not enhance the anti-adenovirus vector immunity of the subject by more than six-fold as compared to that present in the subject before administration, said immunity being determined by hemagglutinin inhibition assay, microneutralization assay, IgA ELISA, and/or ELIspot assay.

42. The method of claim 41, wherein the subject is seropositive for human adenovirus prior to the administration.

43. The method of claim 41, further comprising administering a single dose of a second influenza pharmaceutical formulation about 11-14 months after administration of at least one dose of the previously administered influenza pharmaceutical formulation.

44. The method of claim 43, wherein the second influenza pharmaceutical formulation comprises antigens of a seasonal influenza that are the same or different as that comprised by the previously administered influenza pharmaceutical formulation.

45. (canceled)
